(19) United States
(12) Patent Application Publication
(10) Pub. No.: US 2014/0235630 A1
(43) Pub. Date: Aug. 21, 2014

(54) COMPOSITIONS AND METHODS FOR THE TREATMENT OF PROLIFERATIVE DISEASES

(71) Applicant: Beth Israel Deaconess Medical Center, Inc., Boston, MA (US)

(72) Inventors: Gerburg Wulf, Cambridge, MA (US); Lewis C. Cantley, Cambridge, MA (US)

(21) Appl. No.: 14/348,810

(22) PCT Filed: Sep. 28, 2012
(86) PCT No.: PCT/US2012/057934

§ 371 Date: Mar. 31, 2014

Related U.S. Application Data
(60) Provisional application No. 61/541,964, filed on Sep. 30, 2011.
(60) Provisional application No. 61/683,941, filed on Aug. 16, 2012.

(51) Int. Cl. A61K 31/5377; A61K 31/4439; A61K 31/502

(52) U.S. Cl. CPC A61K 31/5377; A61K 31/502; A61K 31/4439
USPC 514/232.2; 514/248

(57) ABSTRACT

The present invention relates to pharmaceutical compositions that contain PI3K inhibitors (e.g., PBKa-specific inhibitors) and PARP inhibitors. The invention also provides methods for the treatment of proliferative diseases such as cancer (e.g., breast cancer) by administering the composition(s) to a subject.

### BACKGROUND OF THE INVENTION

Proliferative diseases such as cancer are a group of diseases characterized by uncontrolled cellular growth, which can be caused by both external factors (infectious organisms, radiation, chemicals, tobacco, etc.) as well as internal factors (hormones, immune conditions, inherited mutations, etc.). The annual incidence of cancer is estimated to be in excess of 1.5 million in the United States alone. Cancer remains the second-leading cause of death in the U.S., accounting for nearly 1 of every 4 deaths. Worldwide, cancer is also a leading cause of death with an annual incidence of over 10 million.

Breast cancer, in particular, is one of the leading causes of cancer mortality among Western women. An estimated 39,970 breast cancer deaths (39,520 women, 450 men) are expected in 2011 in the United States alone. Over 230,000 new cases of breast cancer are expected to occur in 2011 in the U.S. (with over 2,000 of the new cases expected in men).

The current therapies available for the treatment of cancer (e.g., breast cancer) include surgery, radiation, chemotherapy, and hormone therapy. The therapies are dangerous, costly, toxic, and sometimes ineffective, especially in the treatment of metastatic cancer. Certain metastatic cancers, such as triple-negative breast cancer (TNBC), are especially difficult to address because they are often refractory to standard chemotherapeutic or hormonal treatment. Current treatment options for triple-negative breast cancer are limited to chemotherapeutic regimens that have considerable toxicity and are not curative. Thus, there is a need to develop effective alternative therapies for the treatment of cancer, especially metastatic triple-negative breast cancer and other cancers that are not responsive to conventional cancer therapies.

### SUMMARY OF THE INVENTION

The present invention provides compositions and methods for the treatment of proliferative diseases such as cancer (e.g., triple-negative breast cancer (TNBC)).

In a first aspect, the invention features a pharmaceutical composition that contains a therapeutically effective amount of at least one phosphatidyl inositol 3 kinase (PI3K) inhibitor and at least one poly(ADP-ribose) polymerase (PARP) inhibitor.

In one embodiment of the first aspect, the PI3K inhibitor is a small molecule such as 5-(2,6-dimorpholinopyrimidin-4-yl)-4-(trifluoromethyl)pyridin-2-amine (referenced herein as “Compound A”), (S)-pyrrolidine-1,2-dicarboxylic acid 2-amide 1-[(2-tert-butyl-4′-methyl-[4,5′]bithiazolyl-2′-yl)-amide] (A66 S), [(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5-h]isochromen-10-yl]acetate (PX-866), 2-[(6-aminopurin-9-yl)methyl]-5-methyl-3-(2-methylphenyl)quinazolin-4-one (IC87114), 4-[2-(1H-indazol-4-yl)-6-[(4-methylsulfonylpiperazin-1-yl)methyl]thieno[3,2-d]pyrimidin-4-yl]morpholine (GDC-0941), and/or N-[3-(2,1,3-benzothiadiazol-5-ylamino)quinoxalin-2-yl]-4-methylbenzenesulfonamide (XL147), or a pharmaceutically acceptable salt thereof.

In another embodiment of the first aspect, the PI3K inhibitor is a PI3Ka-specific inhibitor such as (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), 4-[2-(1H-indazol-4-yl)-6-[(4-methylsulfonylpiperazin-1-yl)methyl]thieno[3,2-d]pyrimidin-4-yl]morpholine (GDC-0941), (2S)-1-N-[5-(2-tert-butyl-1,3-thiazol-4-yl)-4-methyl-1,3-thiazol-2-yl]pyrrolidine-1,2-dicarboxamide (A66), N-[(E)-(6-bromoimidazo[1,2-a]pyridin-3-yl)methylideneamino]-N,2-dimethyl-5-nitrobenzenesulfonamide (PIK-75), N-(7,8-dimethoxy-2,3-dihydroimidazo[1,2-c]quinazolin-5-yl)pyridine-3-carboxamide (PIK-90), PWT33597, INK1117, and CNX-1351, or a pharmaceutically acceptable salt thereof. In a preferred embodiment, the PI3Ka-specific inhibitor is (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), or a pharmaceutically acceptable salt thereof.

In another embodiment of the first aspect, the PI3K inhibitor is a pan-class I PI3K inhibitor such as a Compound A-class PI3K inhibitor. Compound A-class PI3K inhibitors include, but are not limited to, 5-(2,6-dimorpholinopyrimidin-4-yl)-4-(trifluoromethyl)pyridin-2-amine (Compound A), (S)-pyrrolidine-1,2-dicarboxylic acid 2-amide 1-[(2-tert-butyl-4′-methyl-[4,5′]bithiazolyl-2′-yl)-amide] (A66 S), [(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5-h]isochromen-10-yl]acetate (PX-866), and 2-[(6-aminopurin-9-yl)methyl]-5-methyl-3-(2-methylphenyl)quinazolin-4-one (IC87114), or a pharmaceutically acceptable salt thereof. In a preferred embodiment, the Compound A-class PI3K inhibitor is 5-(2,6-dimorpholinopyrimidin-4-yl)-4-(trifluoromethyl)pyridin-2-amine (Compound A), or a pharmaceutically acceptable salt thereof. In another preferred embodiment, the Compound A-class PI3K inhibitor is [(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5-h]isochromen-10-yl]acetate (PX-866), or a pharmaceutically acceptable salt thereof.

In another embodiment of the first aspect, the PARP inhibitor is a small molecule. Typically, the PARP inhibitor is 4-[[3-[4-(cyclopropanecarbonyl)piperazine-1-carbonyl]-4-fluorophenyl]methyl]-2H-phthalazin-1-one (i.e., Olaparib, referenced herein as “Compound B”), or a pharmaceutically acceptable salt thereof. Alternatively, or in addition to Compound B, the PARP inhibitor component may include one or more small molecules selected from the following compounds: 4-iodo-3-nitrobenzamide (Iniparib), 2-[(2R)-2-methylpyrrolidin-2-yl]-1H-benzimidazole-4-carboxamide (ABT-888), 8-Fluoro-2-{4-[(methylamino)methyl]phenyl}-1,3,4,5-tetrahydro-6H-azepino[5,4,3-cd]indol-6-one (AG014699), 4-methoxy-carbazole (CEP 9722), 2-[4-[(3S)-piperidin-3-yl]phenyl]indazole-7-carboxamide hydrochloride (MK 4827), and 3-aminobenzamide, or a pharmaceutically acceptable salt thereof.

In a preferred embodiment of the invention, the composition of the invention includes a therapeutically effective amount of (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), or a pharmaceutically acceptable salt thereof, in combination with a therapeutically effective amount of 4-[[3-[4-(cyclopropanecarbonyl)piperazine-1-carbonyl]-4-fluorophenyl]methyl]-2H-phthalazin-1-one (Compound B, i.e., Olaparib), or a pharmaceutically acceptable salt thereof.

In another preferred embodiment of the invention, the composition of the invention includes a therapeutically effective amount of 5-(2,6-dimorpholinopyrimidin-4-yl)-4-(trifluoromethyl)pyridin-2-amine (Compound A), or a pharmaceutically acceptable salt thereof, in combination with a therapeutically effective amount of 4-[[3-[4-(cyclopropanecarbonyl)piperazine-1-carbonyl]-4-fluorophenyl]methyl]-2H-phthalazin-1-one (Compound B, i.e., Olaparib), or a pharmaceutically acceptable salt thereof.

In a second aspect, the invention features a method of treating a subject having a proliferative disease by administering to the subject a therapeutically effective amount of at least one PI3K inhibitor (e.g., PI3Kα-specific inhibitor) and a therapeutically effective amount of at least one PARP inhibitor. The PI3K inhibitor(s) and PARP inhibitor(s), discussed above, may be administered together in the same composition (including any of the compositions of the first aspect of the invention) or in separate compositions or dosage forms. The compositions may be administered intramuscularly, intravenously, intradermally, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularally, orally, topically, locally, by inhalation, injection, infusion, continuous infusion, localized perfusion bathing target cells directly, catheter, lavage, gavage, in cremes, or lipid compositions.

The compositions and methods of the invention may be used to treat any of a wide variety of proliferative diseases that are characterized hyperproliferative conditions such cancer, hyperplasia, fibrosis, angiogenesis, psoriasis, atherosclerosis, and smooth muscle proliferation in the blood vessels. Cancer includes breast cancer, colon adenocarcinoma, esophagas adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, ductal carcinoma, lobular carcinoma, intraductal carcinoma, mucinous carcinoma, phyllodes tumor, Ewing's sarcoma, ovarian cancer, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung cancer, kidney carcinoma, urinary bladder carcinoma, Wilm's tumor, lymphoma, leukemia, and non-Hodgkin's lymphoma.

The compositions and methods of the invention are particularly suitable for treating breast cancers, including advanced breast cancer, metastatic breast cancer, treatment-refractory breast cancer, estrogen receptor (ER)-negative breast cancer, progesterone receptor (PR)-negative breast cancer, human epidermal growth factor receptor 2 (HER2)-negative breast cancer, and/or triple-negative breast cancer (TNBC).

Predisposition to responsiveness of the subject to treatment with the compositions and methods of the invention is determined by detecting an alteration in a germline BRCA1 gene, PTEN expression, BSA1 expression, Akt phosphorylation, and/or H2AX phosphorylation from a sample from the subject relative to a sample from a control subject. A mutation in the germline BRCA1 gene, lower PTEN expression levels, lower BSA1 expression levels, elevated Akt phosphorylation levels, and/or elevated H2AX phosphorylation levels from the sample of the subject relative to the sample of the control subject is indicative of the predisposition to respond to treatment. Preferably, the sample is a bodily fluid of the subject (e.g., blood) or a biopsied tumor tissue.

Typically, the subject is a mammal, such as a human. For breast cancer cases, the human subject is typically a woman.

### I. Definitions

By “phosphatidyl inositol 3 kinase” or “PI3K” is meant a kinase enzyme capable of preferentially phosphorylating phosphoinositides on the 3′ position of the inositol ring. PI3Ks act as signal transducing enzymes involved in cellular functions such as cell growth, proliferation, differentiation, motility, and survival. There are four classes of PI3Ks (I, II, III, and IV), categorized based on structure and substrate specificity. Class I PI3Ks are most closely associated with human disease such as cancer. Class I PI3Ks can be further divided into four different isoforms, PI3Kα, PI3Kβ, PI3Kγ, and PI3Kδ. Of the four isoforms, PI3Kα and PI3Kβ are widely expressed in human tissues and deregulated in many solid tumors.

By “poly(ADP-ribose) polymerase” or “PARP” is meant a protein involved in cellular processes involving mainly DNA repair and programmed cell death. A PARP can be any one of the 17 PARP family members (e.g., PARP1, PARP2, PARP5a, PARP5b). The main role of PARP is to detect single- and double-stranded breaks in DNA and promote repair. PARP utilizes NAD+ as a substrate for generating ADP-ribose monomers, which are then attached to its target substrates (e.g., single-stranded DNA). PARP is inactivated by caspase-3 cleavage during programmed cell death.

By “PI3Kα-specific inhibitor” is meant any compound or compounds capable of preferentially inhibiting PI3Kα over at least one (e.g., one, two, or three) other PI3K class I isoform (PI3Kβ, PI3Kδ, and/or PI3Kγ). For example, a PI3Kα-specific inhibitor is at least two times more potent, preferably at least 5 times more potent, more preferably at least 10 times more potent, against PI3Kα than against PI3Kβ, but may or may not at least two times, at least 5 times, or at least 10 times more potent against PI3Kδ and/or PI3Kγ or other non-class I PI3Ks such as class II PI3Ks (e.g., PI3K-C2α), class III PI3Ks (e.g., Vps34), or class IV PI3Ks (e.g., mTOR or DNA-PK). Preferably, the PI3Kα-specific inhibitor is Compound C, or a related compound, such as those described in WO 2010/029082 (see, e.g., Example 15).

By “pan-class I PI3K inhibitor” is meant any compound or compounds capable of preferentially inhibiting class I PI3Ks over any other kinase enzymes. For example, a pan-class I PI3K inhibitor is at least two times more potent, preferably at least 5 times more potent, more preferably at least 10 times more potent, against class I PI3K enzymes than against other kinases, including the related phosphatidyl inositol 3 kinase-related kinase (PIKK), mammalian target of rapamycin (mTOR). In particular, the PI3K inhibitors may be small molecules or may be biological macromolecules. Typically, the PI3K inhibitors are small molecules, preferably synthetic compounds such as those described in U.S. Patent Application Pub. No. 2010/0249126.

By “PARP inhibitor” is meant any compound or compounds capable of inhibiting PARP. In particular, the PARP inhibitors may be small molecules or may be biological macromolecules. Typically, the PARP inhibitors are small molecules, preferably synthetic compounds such as those described in WO 2004/080976 (i.e., Olaparib).

By “Compound A-class PI3K inhibitor” is meant a pan-class I PI3K inhibitor which preferentially inhibits PI3Kα, PI3Kβ, PI3Kδ, and PI3Kγ over any other kinases, including non-class I PI3K kinases (e.g., class II PI3Ks (e.g., PI3K-C2α), class III PI3K (e.g., Vps34), or class IV PI3K (e.g., mTOR or DNA-PK)). For example, a Compound A-class PI3K inhibitor is at least two times more potent, preferably at least 5 times more potent, more preferably at least 10 times more potent, against PI3Kα, PI3Kβ, PI3Kδ, and PI3Kγ than against other kinases, including phosphatidyl inositol 3 kinase-related kinase (PIKK) and mammalian target of rapamycin (mTOR). Compound A-class inhibitors share similar function and specificity for targets. Typically, the Compound A-class PI3K inhibitors are small molecules, preferably synthetic compounds such as those described in WO 2007/084786 (see, e.g., Example 10).

The term “inhibit” or its grammatical equivalent, such as “inhibiting,” is not intended to require complete reduction in biological activity of a target (e.g., PARP or PI3K). Such reduction is preferably by at least about 50%, at least about 75%, at least about 90%, and more preferably by at least about 95% of the activity of the molecule in the absence of the inhibitory effect, e.g., in the absence of an inhibitor, such as a PARP or pan-class I PI3K inhibitor disclosed in the invention. More preferably, the term refers to an observable or measurable reduction in activity. In treatment scenarios, preferably the inhibition is required to produce a therapeutic benefit in the condition being treated (e.g., cancer).

Throughout this specification and claims, the word “comprise,” or variations such as “comprises” or “comprising,” will be understood to imply the inclusion of a stated integer or group of integers but not the exclusion of any other integer or group of integers.

The terms “effective amount” or “therapeutically effective amount” refer to a sufficient amount of the agent to provide the desired biological, therapeutic, and/or prophylactic result. That result can be reduction and/or alleviation of the signs, symptoms, or causes of a disease (e.g., a proliferative disease such as cancer) or any other desired alteration of a biological system. For example, a “therapeutically effective amount” when used in reference to treating a cancer refers to an amount of one or more compounds that provides a clinically significant decrease in the cancer, e.g., relieves or diminishes one or more symptoms caused by a condition associated with cancer.

A “pharmaceutically acceptable carrier” is meant a carrier which is physiologically acceptable to a treated mammal (e.g., a human) while retaining the therapeutic properties of the compound with which it is administered. One exemplary pharmaceutically acceptable carrier is physiological saline. Other physiologically acceptable carriers and their formulations are known to one skilled in the art and described, for example, in Remington's Pharmaceutical Sciences (Remington's Pharmaceutical Sciences, 21^{th} ed., A. Gennaro, 2005, Lippincott, Williams & Wilkins, Philadelphia, Pa.), incorporated herein by reference.

A “subject” or “host” is a vertebrate, such as a mammal, e.g., a human, specifically a woman. Mammals include, but are not limited to, farm animals (such as cows), sport animals, pets (such as cats, dogs, and horses), mice, rats, and primates.

As used herein, and as well understood in the art, “treatment” is an approach for obtaining beneficial or desired results, such as clinical results. Beneficial or desired results can include, but are not limited to, alleviation or amelioration of one or more symptoms or conditions; diminishment of extent of disease, disorder, or condition; stabilization (i.e., not worsening) of a state of disease, disorder, or condition; prevention of spread of disease, disorder, or condition; delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment. The recitation herein of numerical ranges by endpoints is intended to include all numbers subsumed within that range (e.g., a recitation of 1 to 5 includes 1, 1.5, 2, 2.75, 3, 3.80, 4, and 5).

As used herein, “a” or “an” means at least one or one or more unless otherwise indicated. In addition, the singular forms “a,” “an,” and “the,” include plural referents unless the context clearly dictates otherwise. Thus, for example, reference to “a composition containing a therapeutic agent” includes a mixture of two or more therapeutic agents.

Other features and advantages of the invention will be apparent from the following Detailed Description, the drawings, and the claims.

### BRIEF DESCRIPTION OF THE DRAWINGS

The invention may be more completely understood in consideration of the accompanying drawings, which are incorporated in and constitute a part of this specification, and together with the description, serve to illustrate several embodiments of the invention:

FIGS. 1A-1H are representative images at 400× magnification of immunohistochemistry for phospho-AKT (S473) (1A and 1B), pospho-(Thr202/Tyr204)-ERK (1C and 1D), and the tumor-suppressor phosphatases INPP4B (FIGS. 1E and 1F) and PTEN (FIGS. 1G and 1H), showing PI3K pathway activation in BRCA1-related breast cancer in MMTV-CreBRCA1^{f/f}p53+/− mice. Tumor-bearing females were euthanized, tissues harvested and processed for immunohistochemistry. Adjacent normal mammary gland tissue is on the left (1A, 1C, 1E, and 1G). Tumor tissue on the right (1B, 1D, 1F, and 1H).

FIG. 2A are representative ¹⁸FDG PET-CT scan images of a tumor-bearing mouse at baseline (image on the left) and within 48 hours after start of treatments with the PI3K inhibitor Compound A (50 mg/kg/day by gavage, image on the right). This mouse had developed 4 simultaneous tumors. Arrows are used to identify different tumors upon baseline (left) and post-treatment (right) imaging. The changes in ¹⁸FDG-uptake exhibited a decrease by 45%, 64%, 64%, and 56% for the four tumors.

FIG. 2B are immunohistochemistry (IHC) images of tumor tissue obtained via core needle biopsy before and after two weeks of treatments with Compound A, fixed and processed with anti-pAKT (S473) antibodies, showing suppression of AKT-phosphorylation on 5473 as a result of treatments with Compound A in vivo.

FIG. 2C is a graph showing the relative decrease in FDG-uptake in 6 mammary carcinomas determined by the ratio of uptake at 48 hours or 2 weeks to baseline.

FIG. 2D are ¹⁸FDG PET (upper panels) and CT (lower panels) scan images before (left) and after a 2-week treatment (right) PI3K inhibitor Compound A, showing a concordance of decrease in FDG-uptake and tumor shrinkage. Tumors are marked with arrows in each image. The outline in the CT scan images indicates the tumor circumference before treatment to visualize treatment effect on tumor size.

FIG. 3A are gross pathologic images of an untreated tumor (left), a tumor treated for 2 weeks (middle) and 6 weeks (right) with Compound A at 50 mg/kg/day via gavage.

FIG. 3B is an image showing the level of CD31 by immunohistochemistry in an untreated tumor.

FIG. 3C is an image showing the level of CD31 by immunohistochemistry of the center of a tumor treated for 6 weeks with Compound A.

FIG. 3D is an image showing the level of CD31 by immunohistochemistry of the tumor capsule of a mammary tumor treated for 6 weeks.

FIG. 3E is a graph showing the determination of the Chalkley score to quantify CD31 staining. IHCs with anti-CD31 antibodies were performed in pre-treatment biopsies and in tumor specimen from mice at the time of tumor progression.

FIG. 4A are immunoblots with antibodies against total AKT, EGFR, ERK and their phospho-specific epitopes showing activation of compensatory signaling upon treatment with Compound A in HCC1937 and SUM149 cells. HCC1937 or SUM149 cells were treated with Compound A, Compound B, or its combination as indicated for 72 hours, lysed, and subjected to immunoblotting.

FIG. 4B are immunohistochemical images of pre-treatment biopsies and post-Compound A treatment tumor tissues with antibodies against p-ERK, Ki67, and γH2AX showing an in vivo increase of γH2AX-positive cells after treatment with Compound A and proliferative activity at the “pushing margin.” Tumor-bearing mice were subjected to a pre-treatment biopsy and then treated with Compound A at 50 mg/kg/day.

FIG. 4C are immunoblots with antibodies against PAR, pAKT, γH2AX, Cleaved Caspase 3 (CC3) as an apoptosis marker, and actin following cells treated with Compound A (1 μM) and Compound B (10 μM) or their combination for 24 hours and lysis showing the effects of combined PI3K and PARP inhibition on BRCA1 mutant cells.

FIG. 4D are immunoblots with antibodies against PAR, pAKT, total AKT, γH2AX, and actin following cells treated with Compound A (1 μM) and Compound B (10 μM) or their combination for 24 hours and lysis showing the effects of combined PI3K and PARP inhibition on BRCA1 mutant cells.

FIG. 5A are immunoblots showing the effects of Compound A, ATM inhibitor KU-55933, and their combination on the DNA damage response. HCC1937 were treated for 18 hours with Compound A at 2.5 μM, KU55933 at 10 μM, or their combination, subjected to ionizing irradiation with 10 Gy or mock, lysed 6 hours later, and subjected to immunoblotting.

FIGS. 5B-5E are immunofluorescence images of breast cancer cells isolated from primary tumors from MMTV-CreBRCA1^{f/f}p53+/− mice either treated with vehicle control (5B and 5C) or Compound A (5D and 5E) for 18 hours, followed by irradiation with 10 Gy IR 6 hours later cells, using antibodies against Rad51 and counterstained with DAPI. Rad51 foci are depicted as lighter punctuate signals within the cells (5C). Formation of Rad51 foci in response to ionizing radiation was completely blocked by pre-treatment of these cells with Compound A (5E).

FIG. 5F are immunoblots showing induction of H2AX phosphorylation and poly(ADP)ribosylation (PAR) occur in response to PI3Kα, but not PI3Kβ, inhibition. SUM149 cells were transfected with siRNA pools depleting PI3Kα (left panel) or PI3Kβ (right panel), lysed after 48 hrs, and subjected to immunoblotting with antibodies as indicated.

FIGS. 6A-6D are graphs showing the synergistic effect of a PI3K inhibitor (Compound A) and PARP inhibitor (Compound B) in treating breast cancer. Trendlines (bold lines) were calculated using all data points to determine best fit and show the relative tumor growth in the vehicle control cohort (as in FIG. 6A) and in the Compound A cohort (as in FIG. 6B). The functions of the best-fit curves were used to determine tumor doubling times for all three treatment modalities and controls. Tumor-bearing MMTV-CreBRCA1^{f/f}p53+/− were treated with either vehicle control (6A); Compound A (6B, 50 mg/kg/day (n=11) or 30 mg/kg/day (n=10)); Compound B (6C, 50 mg/kg/day (n=8)); or the combination of Compound A and Compound B (6D, Compound A 50 mg/kg/day+Compound B 50 mg/kg/day (n=8) or Compound A 30 mg/kg/day+Compound B 50 mg/kg/day (n=7)) and tumor volumes were measured every 2-3 days using calipers.

FIG. 6E are immunoblots with antibodies against actin, p-AKT, and γH2AX of tumor tissues harvested from animals 3 hours after last treatment with Compound A (30 mg/kg/day), Compound B (50 mg/kg/day), or their combination.

FIG. 6F is a graph showing the intratumoral Compound A concentrations as assessed by mass spectrometry of tumor tissues harvested from animals 3 hours after last treatment with Compound A (30 mg/kg/day), Compound B (50 mg/kg/day), or their combination.

FIGS. 6G and 6H are graphs showing the relative tumor volume (RTV) over time of tumors from breast cancer tissues from two patients, one with a 185delAG germline mutation (6G) and the other one with a 2080delA germline mutation (6H), propagated as subcutaneous implants in nude mice. Tumors were allowed to grow to a size of 5 mm when mice were randomized to treatments with either vehicle control (squares), Compound A (upside-down triangles), Compound B (triangles), or their combination (diamonds) (n=6 for each cohort, same dosing as in 6F).

FIG. 7 is a graph showing the relative tumor volume (RTV) over time of tumor-bearing MMTV-CreBRCA1^{f/f}p53+/− mice treated with a control vehicle (n=3), PI3Kα-specific inhibitor Compound C alone (n=7), or a combination of pan-PARP inhibitor Compound B and PI3Kα-specific inhibitor Compound C (n=6). The data of the control and Compound C alone treatment groups are represented by trendlines calculated using all data points to determine best fit. The data of the combination treatment group are individually represented.

FIG. 8A are immunohistochemical images of tumor tissues obtained from tumor-bearing MMTV-CreBRCA1^{f/f}p53+/− mice pre-treatment (left column of images), at Day 10 of treatment (middle column of images), and post-treatment tissue at the time of progression (right column of images) following treatment with a combination of Compound A and Compound B.

FIG. 8B is a graph of Ki67 (right bar graph in each treatment group) and γH2AX (left bar graph in each treatment group) levels in tumor biopsies in pre-treatment, on-treatment, and post-treatment at the time of progression groups. Ki67 and γH2AX were scored by counting and averaging the number of positive nuclei per high-power field (HPF).

FIG. 8C is a graph showing stable body mass with PI3K-inhibitor and PARP-inhibitor treatments. Mice were weighed before, during treatment, and after completion of treatments.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF THE INVENTION

The present invention is based, at least in part, on the discovery that the combination of a PI3K inhibitor (e.g., a PI3Kα-specific inhibitor or a pan-class I PI3K inhibitor) and pan-PARP inhibitor is effective in treating certain proliferative diseases such as cancer, including breast cancer (e.g., triple-negative breast cancer) that is not responsive to other forms of treatment. Furthermore, the data presented herein show that these compounds, when used together, have unexpected, synergistic effects.

I. Phosphatidyl inositol 3 kinases (PI3Ks)

The compositions and methods of the present invention make use of one or more PI3K inhibitors. Protein kinases, such as PI3Ks, belong to a large and diverse family of enzymes that catalyze protein phosphorylation and play a critical role in cellular signaling. Protein kinases may exert positive or negative regulatory effects, depending upon their target protein. Protein kinases are involved in specific signaling pathways which regulate cell functions such as, but not limited to, metabolism, cell cycle progression, cell adhesion, vascular function, apoptosis, and angiogenesis. Malfunctions of cellular signaling have been associated with many proliferative diseases, including cancer (e.g., breast cancer).

Phosphatidyl inositol 3 kinases (PI3Ks) comprise a family of lipid and serine/threonine kinases that catalyze the transfer of phosphate to the D-3′ position of inositol lipids to produce phosphoinositol-3-phosphate (PIP), phosphoinositol-3,4-diphosphate (PIP2), and phosphoinositol-3,4,5-triphosphate (PIP3). The PIPs, in turn, act as second messengers in signaling cascades by docking proteins containing pleckstrin-homology, FYVE, Phox, and other phospholipid-binding domains into a variety of signaling complexes often at the plasma membrane (Vanhaesebroeck et al., Annu. Rev. Biochem., 70: 535, 2001; Katso et al., Annu. Rev. Cell Dev. Biol., 17: 615, 2001). Of the two class I PI3Ks, class Ia PI3Ks are heterodimers composed of a catalytic p110 subunit (α, β, and δ isoforms) constitutively associated with a regulatory subunit that can be p85α, p55α, p50α, p85β, or p55γ. The class Ib sub-class has one family member, a heterodimer composed of a catalytic p110γ subunit associated with one of two regulatory subunits, p101 or p84 (Frumanet et al., Annu Rev. Biochem., 67: 481, 1998; Suire et al., Curr. Biol., 15: 566, 2005). The modular domains of the p85/55/50 subunits include Src Homology (SH2) domains that bind phosphotyrosine residues in a specific sequence context on activated receptor and cytoplasmic tyrosine kinases, resulting in activation and localization of Class 1a PI3Ks. Class Ib PI3K is activated directly by G protein-coupled receptors that bind a diverse repertoire of peptide and non-peptide ligands (Stephens et al., Cell, 89: 105, 1997; Katso et al., Annu. Rev. Cell Dev. Biol., 17: 615-675, 2001). Consequently, the resultant phospholipid products of class I PI3K link upstream receptors with downstream cellular activities including proliferation, survival, chemotaxis, cellular trafficking, motility, metabolism, inflammatory and allergic responses, transcription, and translation (Cantley et al., Cell, 64: 281, 1991; Escobedo and Williams, Nature, 335: 85, 1988; Fantl et al., Cell, 69: 413, 1992).

In many cases, PIP2 and PIP3 recruit Akt, the product of the human homologue of the viral oncogene v-Akt, to the plasma membrane where it acts as a nodal point for many intracellular signaling pathways important for growth and survival (Fantl et al., Cell, 69: 413-423, 1992; Bader et al., Nat. Rev. Cancer, 5: 921, 2005; Vivanco and Sawyer, Nat. Rev. Cancer, 2: 489, 2002). Aberrant regulation of PI3K, which often increases survival through Akt activation, is one of the most prevalent events in human cancer and has been shown to occur at multiple levels. The tumor suppressor gene PTEN, which dephosphorylates phosphoinositides at the 3′ position of the inositol ring and in so doing antagonizes PI3K activity, is functionally deleted in a variety of tumors. In other tumors, the genes for the p110α isoform, PIK3CA, and for Akt are amplified, and increased protein expression of their gene products has been demonstrated in several human cancers. Furthermore, mutations and translocation of p85α that serve to up-regulate the p85-pl 10 complex have been described in a few human cancers. Finally, somatic missense mutations in PIK3CA that activate downstream signaling pathways have been described at significant frequencies in a wide diversity of human cancers (Kang et al., Proc. Natl. Acad. Sci. USA, 102: 802, 2005; Samuels et al., Science, 304: 554, 2004; Samuels et al., Cancer Cell, 7: 561-573, 2005). These observations show that deregulation of PI3K and the upstream and downstream components of this signaling pathway is one of the most common deregulations associated with human proliferative diseases such as cancers.

Suitable PI3K inhibitors for use in the compositions and methods of the invention include, but are not limited to, PI3Kα-specific inhibitors or pan-class I PI3K inhibitors, which include Compound A-class PI3K inhibitors. PI3Kα-specific inhibitors include, but are not limited to, (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), 4-[2-(1H-indazol-4-yl)-6-[(4-methylsulfonylpiperazin-1-yl)methyl]thieno[3,2-d]pyrimidin-4-yl]morpholine (GDC-0941), (2S)-1-N-[5-(2-tert-butyl-1,3-thiazol-4-yl)-4-methyl-1,3-thiazol-2-yl]pyrrolidine-1,2-dicarboxamide (A66), N-[(E)-(6-bromoimidazo[1,2-a]pyridin-3-yl)methylideneamino]-N,2-dimethyl-5-nitrobenzenesulfonamide (PIK-75), N-(7,8-dimethoxy-2,3-dihydroimidazo[1,2-c]quinazolin-5-yl)pyridine-3-carboxamide (PIK-90), PWT33597, INK1117, and CNX-1351, or a pharmaceutically acceptable salt thereof. Other PI3Kα-specific inhibitors are described in U.S. Pub. Nos. 2011/0230476, 2009/0312319, 2011/0281866, 2011/0269779, 2010/0249099, and 2011/0009405, each of which is incorporated herein by reference. The chemical structure of Compound C is:

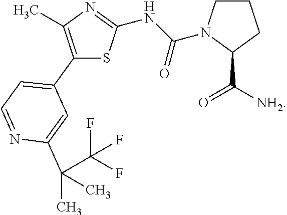 [SMILES: Cc1nc(NC(=O)N2CCC[C@H]2C(N)=O)sc1-c1ccnc(C(C)(C)C(F)(F)F)c1]

Compound A-class PI3K inhibitors include, but are not limited to, 5-(2,6-dimorpholinopyrimidin-4-yl)-4-(trifluoromethyl)pyridin-2-amine (Compound A), (S)-pyrrolidine-1,2-dicarboxylic acid 2-amide 1-[(2-tert-butyl-4′-methyl-[4,5′]bithiazolyl-2′-yl)-amide] (A66 S), [(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5-h]isochromen-10-yl]acetate (PX-866), and/or 2-[(6-aminopurin-9-yl)methyl]-5-methyl-3-(2-methylphenyl)quinazolin-4-one (IC87114), or a pharmaceutically acceptable salt thereof. Other suitable PI3K inhibitors for use in the invention include, but are not limited to, pan class I PI3K inhibitors 4-[2-(1H-indazol-4-yl)-6-[(4-methylsulfonylpiperazin-1-yl)methyl]thieno[3,2-d]pyrimidin-4-yl]morpholine (GDC-0941) and/or N-[3-(2,1,3-benzothiadiazol-5-ylamino)quinoxalin-2-yl]-4-methylbenzenesulfonamide (XL147), or a pharmaceutically acceptable salt thereof. The chemical structure of Compound A is:

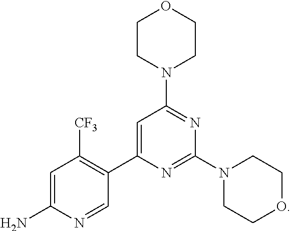 [SMILES: Nc1cc(C(F)(F)F)c(-c2cc(N3CCOCC3)nc(N3CCOCC3)n2)cn1]

### II. Poly(ADP-Ribose) Polymerase (PARP)

The compositions and methods of the invention also make use of one or more PARP inhibitors. The poly (ADP-ribose) polymerase (PARP) is also known as poly (ADP-ribose) synthase and poly ADP-ribosyltransferase. PARP catalyzes the formation of mono- and poly (ADP-ribose) polymers which can attach to cellular proteins (as well as to itself) and thereby modify the activities of those proteins. The enzyme plays a role in regulation of transcription, cell proliferation, and chromatin remodeling (see D'amours et al., Biochem., 342: 249268, 1999).

PARP comprises an N-terminal DNA binding domain, an automodification domain, and a C-terminal catalytic domain. Various cellular proteins interact with PARP. The N-terminal DNA binding domain contains two zinc finger motifs. Transcription enhancer factor-1 (TEF-1), retinoid X receptor α, DNA polymerase α, X-ray repair cross-complementing factor-1 (XRCC1) and PARP itself interact with PARP in this domain. The automodification domain contains a BRCT motif, one of the protein-protein interaction modules. This motif is originally found in the C-terminus of BRCA1 (breast cancer susceptibility protein 1) and is present in various proteins related to DNA repair, recombination and cell-cycle checkpoint control. POU-homeodomain-containing octamer transcription factor-1 (Oct-1), YinYang (YY)1, and ubiquitinconjugating enzyme 9 (ubc9) could interact with this BRCT motif in PARP.

More than 15 members of the PARP family of genes are present in the mammalian genome. PARP family proteins and poly(ADP-ribose) glycohydrolase (PARG), which degrades poly(ADP-ribose) to ADP-ribose, are involved in a variety of cell regulatory functions including DNA damage response and transcriptional regulation and associated with carcinogenesis and the biology of cancer.

Several PARP family proteins have been identified. Tankyrase has been found as an interacting protein of telomere regulatory factor 1 (TRF-1) and is involved in telomere regulation. Vault PARP (VPARP) is a component in the vault complex, which acts as a nuclear-cytoplasmic transporter. PARP-2, PARP-3 and 2,3,7,8-tetrachlorodibenzo-p-dioxin inducible PARP (TiPARP) have also been identified. Therefore, poly(ADP-ribose) metabolism could be related to a variety of cell regulatory functions.

A member of this gene family is PARP-1. The PARP-1 gene product is expressed at high levels in the nuclei of cells and is dependent upon DNA damage for activation. It is believed that PARP-1 binds to DNA single or double-stranded breaks (DSBs) through an amino-terminal DNA-binding domain. The binding activates the carboxy-terminal catalytic domain and results in the formation of polymers of ADP-ribose on target molecules. PARP-1 is itself a target of poly ADP-ribosylation by virtue of a centrally located automodification domain. The ribosylation of PARP-1 causes dissociation of the PARP-1 molecules from the DNA. The entire process of binding, ribosylation, and dissociation occurs very rapidly. It has been suggested that this transient binding of PARP-1 to sites of DNA damage results in the recruitment of DNA repair machinery or may act to suppress the recombination long enough for the recruitment of repair machinery.

The source of ADP-ribose for the PARP reaction is nicotinamide adenosine dinucleotide (NAD). NAD is synthesized in cells from cellular ATP stores and thus high levels of activation of PARP activity can rapidly lead to depletion of cellular energy stores. It has been demonstrated that induction of PARP activity can lead to cell death that is correlated with depletion of cellular NAD and ATP pools. PARP activity is induced in many instances of oxidative stress or during inflammation. For example, during reperfusion of ischemic tissues reactive nitric oxide is generated and nitric oxide results in the generation of additional reactive oxygen species including hydrogen peroxide, peroxynitrate, and hydroxyl radical. These latter species can directly damage DNA and the resulting damage induces activation of PARP activity. Frequently, it appears that sufficient activation of PARP activity occurs such that the cellular energy stores are depleted and the cell dies. A similar mechanism is believed to operate during inflammation when endothelial cells and pro-inflammatory cells synthesize nitric oxide, which results in oxidative DNA damage in surrounding cells and the subsequent activation of PARP activity. The cell death that results from PARP activation is believed to be a major contributing factor in the extent of tissue damage that results from ischemia-reperfusion injury or from inflammation.

PARP (poly-ADP ribose polymerase) participates in a variety of DNA-related functions including cell proliferation, differentiation, apoptosis, DNA repair and also effects on telomere length and chromosome stability (d'Adda di Fagagna et al., Nature Gen., 23(1): 76-80, 1999). Oxidative stress-induced overactivation of PARP consumes NAD+ and consequently ATP, culminating in cell dysfunction or necrosis. This cellular suicide mechanism has been implicated in the pathomechanism of cancer, stroke, myocardial ischemia, diabetes, diabetes-associated cardiovascular dysfunction, shock, traumatic central nervous system injury, arthritis, colitis, allergic encephalomyelitis, and various other forms of inflammation. PARP has also been shown to associate with and regulate the function of several transcription factors.

Suitable PARP inhibitors for use in the compositions and methods of the invention include, but are not limited to, 4-[[3-[4-(cyclopropanecarbonyl)piperazine-1-carbonyl]-4-fluorophenyl]-methyl]-2H-phthalazin-1-one (Compound B, i.e., Olaparib), 4-iodo-3-nitrobenzamide (Iniparib), 2-[(2R)-2-methylpyrrolidin-2-yl]-1H-benzimidazole-4-carboxamide (ABT-888), 8-Fluoro-2-{4-[(methylamino)methyl]-phenyl}-1,3,4,5-tetrahydro-6H-azepino[5,4,3-cd]indol-6-one (AG014699), 4-methoxy-carbazole (CEP 9722), 2-[4-[(3S)-piperidin-3-yl]phenyl]indazole-7-carboxamide hydrochloride (MK 4827), and 3-aminobenzamide, or a pharmaceutically acceptable salt thereof.

### III. Methods of Treatment of a Proliferative Disease Using Compositions of the Invention

The compositions and methods of the invention can be used for treating a subject with a proliferative disease such as cancer. In particular, the compositions of the invention can be used to treat a subject with breast cancer, colon adenocarcinoma, esophagas adenocarcinoma, liver hepatocellular carcinoma, squamous cell carcinoma, pancreas adenocarcinoma, islet cell tumor, rectum adenocarcinoma, gastrointestinal stromal tumor, stomach adenocarcinoma, adrenal cortical carcinoma, follicular carcinoma, papillary carcinoma, ductal carcinoma, lobular carcinoma, intraductal carcinoma, mucinous carcinoma, phyllodes tumor, Ewing's sarcoma, ovarian cancer, endometrium adenocarcinoma, granulose cell tumor, mucinous cystadenocarcinoma, cervix adenocarcinoma, vulva squamous cell carcinoma, basal cell carcinoma, prostate adenocarcinoma, giant cell tumor of bone, bone osteosarcoma, larynx carcinoma, lung cancer, kidney carcinoma, urinary bladder carcinoma, Wilm's tumor, lymphoma, leukemia, and non-Hodgkin's lymphoma.

Preferably, the compositions of the invention can be used to treat a subject with advanced breast cancer, metastatic breast cancer, treatment-refractory breast cancer, estrogen receptor (ER)-negative breast cancer, progesterone receptor (PR)-negative breast cancer, human epidermal growth factor receptor 2 (HER2)-negative breast cancer, or triple-negative breast cancer (TNBC).

Despite the availability of treatments for breast cancer (e.g., radiation, chemotherapy, surgery, hormone therapy), some forms of breast cancers are not amenable to the current available treatments. For example, TNBC remains an incurable illness that invariably relapses after what is considered standard care (e.g., mono- or poly-chemotherapy with anthracyclines, alkylating agents, taxanes, or platinum drugs). In a metastatic setting, relapse is inevitable after these treatments and occurs within months. Targeted treatments, such as vascular endothelial growth factor receptor (VEGFR) and epidermal growth factor receptor (EGFR) inhibitors, have also not improved survival for subjects with certain forms breast cancer, e.g., TNBC.

Specifically, the present invention relates to methods of treating a subject with a proliferative disease (e.g., cancer, e.g., breast cancer, e.g., TNBC) by administration of both a PI3K inhibitor (e.g., PI3Kα-specific inhibitor or pan-class I PI3K inhibitor, e.g., Compound A-class PI3K inhibitor) and a PARP inhibitor (e.g., Compound B) (e.g., concurrent administration of both PI3K inhibitor and PARP inhibitor, either as a single composition or separate compositions). PI3K inhibitors and PARP inhibitors act synergistically to treat the proliferative disease with significantly improved outcome over treatment of the disease with either inhibitor alone. The present invention also relates to methods of treating a subject with a proliferative disease (e.g., cancer, e.g., breast cancer, e.g., TNBC) by the individual administration of a PI3K inhibitor (e.g., PI3Kα-specific inhibitor or pan-class I PI3K inhibitor, e.g., Compound A-class PI3K inhibitor) or a PARP inhibitor (e.g., Compound B).

### IV. Pharmaceutical Formulation and Administration of the Compositions of the Invention

### 1. Administration

The compositions of the invention can be administered to a subject (e.g., a human, e.g., a woman) to treat, prevent, ameliorate, inhibit the progression of, or reduce the severity of one or more symptoms of a proliferative disease (e.g., cancer, e.g., breast cancer, e.g., TNBC) in the subject. Examples of the symptoms of, e.g., cancer that can be treated using the compositions of the invention include, e.g., fatigue, weight change, skin change, persistent coughing, changes in bowel or bladder habits, difficulty swallowing, hoarseness, persistent indigestion after eating, persistent and unexplained muscle or joint pain, fever, headache, chills, diarrhea, vomiting, rash, dizziness, seizures, organ failure, personality changes, confusion. These symptoms, and their resolution during treatment, may be measured by, e.g., a physician during a physical examination or by other tests and methods known in the art.

When treating a subject having a proliferative disease, the at least one PI3K inhibitor (e.g., Compound C or Compound A) and the at least one PARP inhibitor (e.g., Compound B) are administered together in the same composition (e.g., a fixed dosage form) or separately in separate compositions (e.g., non-fixed/separate dosage forms) in an amount sufficient to treat the subject. Accordingly, the compositions may be administered simultaneously, separately, or sequentially.

The compositions utilized in the methods described herein can be formulated for administration by a route selected from, e.g., parenteral, dermal, transdermal, ocular, inhalation, buccal, sublingual, perilingual, nasal, rectal, topical administration, and oral administration. Parenteral administration includes intravenous, intraperitoneal, subcutaneous, and intramuscular administration. Parenteral, intranasal, or intraocular administration may be provided by using, e.g., aqueous suspensions, isotonic saline solutions, sterile and injectable solutions containing pharmacologically compatible dispersants and/or solubilizers, for example, propylene glycol or polyethylene glycol, lyophilized powder formulations, and gel formulations. The preferred method of administration can vary depending on various factors (e.g., the components of the composition being administered and the severity of the condition being treated). Formulations suitable for oral or nasal administration may consist of liquid solutions, such as an effective amount of the composition dissolved in a diluent (e.g., water, saline, or PEG-400), capsules, sachets, tablets, or gels, each containing a predetermined amount of the composition of the invention. The pharmaceutical composition may also be an aerosol formulation for inhalation, e.g., to the bronchial passageways. Aerosol formulations may be mixed with pressurized, pharmaceutically acceptable propellants (e.g., dichlorodifluoromethane, propane, or nitrogen). In particular, administration by inhalation can be accomplished by using, e.g., an aerosol containing sorbitan trioleate or oleic acid, for example, together with trichlorofluoromethane, dichlorofluoromethane, dichlorotetrafluoroethane, or any other biologically compatible propellant gas.

Pharmaceutical compositions according to the invention described herein may be formulated to release the composition immediately upon administration (e.g., targeted delivery) or at any predetermined time period after administration using controlled or extended release formulations. Administration of the pharmaceutical composition in controlled or extended release formulations is useful where the composition, either alone or in combination, has (i) a narrow therapeutic index (e.g., the difference between the plasma concentration leading to harmful side effects or toxic reactions and the plasma concentration leading to a therapeutic effect is small; generally, the therapeutic index, TI, is defined as the ratio of median lethal dose (LD₅₀) to median effective dose (ED₅₀)); (ii) a narrow absorption window at the site of release; or (iii) a short biological half-life, so that frequent dosing during a day is required in order to sustain a therapeutic level.

Many strategies can be pursued to obtain controlled or extended release in which the rate of release outweighs the rate of metabolism of the pharmaceutical composition. For example, controlled release can be obtained by the appropriate selection of formulation parameters and ingredients, including, e.g., appropriate controlled release compositions and coatings. Suitable formulations are known to those of skill in the art. Examples include single or multiple unit tablet or capsule compositions, oil solutions, suspensions, emulsions, microcapsules, microspheres, nanoparticles, patches, and liposomes.

The compositions of the invention may be administered to provide treatment to a subject having cancer, such as breast cancer. The composition may be administered to the subject, e.g., 1, 2, 3, 4, 5, 6, 7, 8, 9, 10, 15, 20, 30, 35, 40, 45, 50, 55, or 60 minutes, 2, 4, 6, 10, 15, or 24 hours, 2, 3, 5, or 7 days, 2, 4, 6 or 8 weeks, 3, 4, 6, or 9 months, 1, 2, 3, 4, 5, 6, 7, 8, 9, 10, 15, 20 years or longer post-diagnosis of cancer.

When treating a proliferative disease (e.g., breast cancer, e.g., TNBC), the compositions of the invention may be administered to the subject either before the occurrence of symptoms or a definitive diagnosis or after diagnosis or symptoms become evident. For example, the composition may be administered, e.g., immediately after diagnosis or the clinical recognition of symptoms or 2, 4, 6, 10, 15, or 24 hours, 2, 3, 5, or 7 days, 2, 4, 6 or 8 weeks, or even 3, 4, or 6 months after diagnosis or detection of symptoms.

The compositions may be sterilized by conventional sterilization techniques, or may be sterile filtered. The resulting aqueous solutions may be packaged for use as is, or lyophilized, the lyophilized preparation may be administered in powder form or combined with a sterile aqueous carrier prior to administration. The pH of the preparations typically will be between 3 and 11, more preferably between 5 and 9 or between 6 and 8, and most preferably between 7 and 8, such as 7 to 7.5. The resulting compositions in solid form may be packaged in multiple single dose units, each containing a fixed amount of the PI3K inhibitor (e.g., PI3Kα-specific inhibitor or pan-class I PI3K inhibitor, e.g., Compound A-class PI3K inhibitor) and PARP inhibitor (e.g., Compound B) and, if desired, one or more agents, such as in a sealed package of tablets or capsules, or in a suitable dry powder inhaler (DPI) capable of administering one or more doses.

### 2. Dosages

The dose of the compositions of the invention or the number of treatments using the compositions of the invention may be increased or decreased based on the severity of, occurrence of, or progression of, the proliferative disease in the subject (e.g., based on the severity of one or more symptoms of, e.g., breast cancer), but generally range from about 0.5 mg to about 3,000 mg of each agent per dose one or more times per week (e.g., 2, 3, 4, 5, 6, or 7 or more times per week).

The pharmaceutical compositions of the invention can be administered in a therapeutically effective amount that provides a protective effect against the proliferative disease (e.g., cancer). The dosage administered depends on the subject to be treated (e.g., the age, body weight, capacity of the immune system, and general health of the subject being treated), the form of administration (e.g., as a solid or liquid), the manner of administration (e.g., by injection, inhalation, dry powder propellant), and the cells targeted (e.g., epithelial cells, such as blood vessel epithelial cells, nasal epithelial cells, or pulmonary epithelial cells). The composition is preferably administered in an amount that provides a sufficient level of PI3K and PARP inhibitors that reduces or prevents one or more symptoms of, e.g., cancer, without undue adverse physiological effects in the subject caused by the treatment.

In addition, single or multiple administrations of the compositions of the present invention may be given to a subject with a proliferative disease (e.g., one administration or administration two or more times). Responsiveness of subjects treated by the pharmaceutical compositions described herein may be measured by, e.g., a physician during a physical examination or by other tests and methods known in the art, e.g., by measuring tumor cell glucose uptake by fluorodeoxyglucose-positron emission tomography (FDG-PET). The dosages may then be adjusted or repeated as necessary.

A single dose of the compositions of the invention may reduce, treat, or prevent one or more symptoms of the cancer in the subject. In addition, a single dose of the compositions of the invention can also be used to achieve therapy in subjects being treated for a cancer. Multiple doses (e.g., 2, 3, 4, 5, or more doses) can also be administered, in necessary, to these subjects.

### 3. Carriers, Excipients, Diluents

The compositions of the invention include PI3K inhibitors (e.g., PI3Kα-specific inhibitors, e.g., Compound C, or pan-class I PI3K inhibitors, e.g., Compound A-class PI3K inhibitors) and PARP inhibitors (e.g., Compound B). Therapeutic formulations of the compositions of the invention are prepared using standard methods known in the art by mixing the active ingredient having the desired degree of purity with optional physiologically acceptable carriers, excipients, or stabilizers (Remington's Pharmaceutical Sciences, 21^{th} ed., A. Gennaro, 2005, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, but preferably, the formulation contains a pharmaceutically acceptable salt, preferably sodium chloride, and preferably at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

### EXAMPLES

The following examples are to illustrate the invention. They are not meant to limit the invention in any way.

### Example 1

### Materials and Methods

### Materials

The PI3K inhibitor 5-(2,6-dimorpholinopyrimidin-4-yl)-4-(trifluoromethyl)pyridin-2-amine (Compound A) was obtained through a Material Transfer Agreement with Novartis Pharmaceuticals. Olaparib (Compound B) was purchased from LC Laboratories (Woburn, Mass.) and KU-55933 was purchased from Selleck Chemicals (Houston, Tex.). BRCA1-mutant human breast cancer cell line HCC1937 was from American Type Culture Collection; # CRL-2336, and maintained in DMEM/10% FBS and SUM149 was from Division of Signal Transduction, BIDMC, maintained in Ham's F-12 with 5% fetal bovine serum (FBS), 5 μg/ml insulin, 2 μg/ml hydrocortisone, 5 μg/ml gentamicin and 2.5 μg/ml fungizone. Cell lines were authenticated by immunoblotting for BRCA1 and PTEN and tested for absence of mycoplasma.

### Animal Experimentation

All animal experiments were conducted in accordance with IACUC-approved protocols at Beth Israel Deaconess Medical Center, Boston, and at the University of Vall d'Hebron, Barcelona, Spain. Female MMTV-CreBRCA1^{f/f}p53+/− mice were obtained by breeding BRCA1 conditional knockout mice from the NIH repository (01XC8, strain C57BL/6) (Xu et al. Nat. Genet., 22: 37-43, 1999); with MMTV-Cre (Jackson Laboratory B6129-TgN(MMTV-Cre)4Mam) (Wagner et al. Nucleic Acids Res., 25: 4323-4330, 1997) and p53 knockout (Taconic Farms, P53N12-M, C57BL/6) (Donehower et al. Nature, 356: 215-221, 1992). At the time of the study, mice had been inbred for 4 years (>7 generations). The floxed or wild type status of Brca1, the presence of the MMTV-Cre transgene and the p53 heterozygosity were determined by PCR. Mice were examined for the occurrence of tumors twice weekly. When tumormetrics were performed, the length and width of the tumor was determined using calipers, and the tumor volume was determined (width²×length/2). Tumor volume was used as a measure of growth and was recorded as ratio to tumor volume at diagnosis. Tumor doubling times were calculated using the functions of the best fit curves for all data points in each treatment modality. 5-(2,6-dimorpholinopyrimidin-4-yl)-4-(trifluoromethyl)pyridin-2-amine (Compound A) was resuspended in 5% Methylcellulose solution (Fluka) and administered via oral gavage at 50 mg/kg/day or 30 mg/kg/day. 4-[[3-[4-(cyclopropanecarbonyl)piperazine-1-carbonyl]-4-fluorophenyl]methyl]-2H-phthalazin-1-one (Compound B) was resuspended for intraperitoneal administration as described (Rottenberg et al. Proc. Natl. Acad. Sci. USA., 105: 17079-17084, 2008) and dosed at 50 mg/kg/day. For patient-derived tumor grafts consent for tumor use was obtained from patients under a protocol approved by the Vall d'Hebron Hospital Clinical Investigation Ethical Committee. Tumors were subcutaneously implanted in 6-week-old female HsdCpb:NMRI-Foxn1nu mice (Harlan Laboratories, Italy) Animals were supplemented with 1 μM estradiol (Sigma) in the drinking water. After tumor graft growth, tumor tissue was re-implanted into recipient mice, which were randomized upon implant growth.

### FDG-PET-Scanning

0.3 to 0.4 mCi of fluorine-18-deoxyglucose were injected intravenously through the retroorbital vein of the anesthetized mouse. After a “washout” period of 1 hour the mouse was imaged on a NanoPET/CT (Bioscan/Mediso) scanner. The NanoPET/CT is a high-resolution small-animal multimodality scanner consisting of 12 lutetium yttrium oxyorthosilicate (LYSO) detector blocks. The blocks comprise a total of 39,780 crystals each with a dimension of 1.2×1.2×13 mm³.

Images were acquired in three dimensions. The mice remained supine and maintained their position throughout the procedure. First, a CT scan was performed and second, a whole-body ¹⁸F-FDG PET emission scan was acquired covering the same area as the CT scan. Counts per minute (cpm) were obtained, converted to mCi, and values were normalized for ROI volume and injected dose. In order to correct for metabolic variability between exams and to determine tumor-specific uptake changes, FDG-uptake rates were corrected for cardiac FDG-uptake (μCi injected/voxels^{tumor})/(μCi/μCi injected/voxels^{heart}). For studies involving repeat scanning, the change in tumor-specific FDG-uptake was determined in percent (1−(FDG-uptake^{post}/FDG-uptake^{pre})×100) Animals were housed in the Longwood SAIF satellite animal facility between scans.

### Immunohistochemistry

For immunohistochemistry we used anti-cleaved caspase 3 (CC3) (9661S, Cell Signalling, Rabbit polyclonal Asp175), anti-Ki67 (9106-S; Thermo Scientific, Rabbit monoclonal SP6). All other antibodies used are described in the immunoblotting section below. All immunohistochemistries were done as described previously (Burga et al. Breast Cancer Res., 13:R30, 2011) including antigen retrieval with a citrate-buffer.

### Immunoblotting

Cells were treated with mock, 5-(2,6-dimorpholinopyrimidin-4-yl)-4-(trifluoromethyl)pyridin-2-amine (Compound A), 4-[[3-[4-(cyclopropanecarbonyl)piperazine-1-carbonyl]-4-fluorophenyl]methyl]-2H-phthalazin-1-one (Compound B), KU-55933, or the combination and lysed in cell lysis buffer (9803, Cell Signaling) as per the manufacturer's instructions. Immunoblots were performed using the Nupage System (Invitrogen). The protein concentration was determined using Bradford reagent (Biorad). A total of 20 μg of protein were loaded, except for PAR, Phospho ATM and Phospho DNA-PK/PRKDC western blots, where 40 μg were loaded. Tumor tissue lysates were prepared similarly with the exception of tissue homogenization by using an electric homogenizer for 30 secs after addition of the lysis buffer. Primary antibodies used for western blotting were total AKT (9272), Cleaved Caspace 3 (9661), total ERK (4695), Phospho AKT Ser473 (4058), Phospho ERK Thr202/Tyr204 (9106), Phospho-Histone H2AX Ser139 (2577), PTEN (9559) from Cell Signaling. Phospho ATM Ser1981 (2152-1), Phospho DNA-PK/PRKDC Ser2056 (3892-1) from Epitomics, Inc. CD31 (ab28364), Actin (ab6276), INPP4B (ab81269) from Abcam; pADPr (sc56198) from Santacruz Biotechnology; and Ki-67 (RM-9106) was purchased from Thermo Scientific.

### Immunofluorescence

Cells were plated on coverslips in a 6-well plates and incubated overnight at 37° C. with 5% CO₂ before drug treatment. Cells were exposed to 5-(2,6-dimorpholinopyrimidin-4-yl)-4-(trifluoromethyl)pyridin-2-amine (Compound A) for 24 hrs followed by irradiation (10 Gy). Cells were fixed with 3% paraformaldehyde and 2% sucrose diluted in PBS 6 h post-irradiation and subsequently permeabilized with 0.5% TritonX-100 buffer (20 mM HEPES pH7.4, 50 mM NaCl, 3 mM MgCl, 300 mM sucrose) for 3 minutes on ice. Cells were incubated with a primary rabbit anti human Rad-51 antiserum at 1:500 dilution in hybridization buffer (5% goat serum, 0.5% NaN3, 1×PBS) for 30 min at 37° C. and subsequently washed thrice in PBS before incubation with secondary antibody for 30 minutes at 37° C. Secondary antibody used was a donkey anti-rabbit Alexafluor 488 conjugated (Invitrogen) at a concentration of 1:50. Cells were again washed thrice with PBS before mounting on slides (25×75×1 mm, Fisherbrand) using Prolong Gold antifade reagent with DAPI (Invitrogen). The edges of the coverglasses were sealed with clear nail polish. From the step using the secondary antibody onward, all procedures were performed in the dark. Images were acquired using a Zeiss 710 NLO laser scanning confocal microscope.

siRNA Transfections

The siRNAs were obtained from Dharmacon, Lafayette. SUM149 cells were transfected with either 10 or 30 nM pool of 4 siRNA sequences targeting PIK3CA (cat#L-003018-00-0005) or PIK3CB (cat#L-003019-00-0005) siRNA using HiPerFect Transfection Reagent (QIAGEN) according to the manufacturer's protocol. Control cells were treated with HiPerFect alone. Cells were grown and harvested 48 h after the transfection using cell lysis buffer (9803, Cell Signaling) as per the manufacturer's instructions and analyzed by Immunoblotting.

### Cell Viability Assay

For cell viability assays, breast cancer cells were seeded at a density of 250 cells/well in 96-well plates in the absence or presence of drugs, and cell viability was determined using the CellTiter-Glo Luminescent Cell Viability Assay (Promega, Madison, Wis., USA) according to the manufacturer's instructions, using a Wallac 3 plate reader.

### Sequencing

Genomic DNA was isolated and PCR amplification performed for regions in the murine PI3K gene that are homologous to the regions frequently mutated in human breast cancer, i.e., E542K and E545K in the helical domain and H1047R in the kinase domain. Primers used were for:

| Exon 9: | |
|---|---|
| (SEQ ID NO: 1) | |
| Forward | CGCATACCTGCATCTGTTCTA; |
| (SEQ ID NO: 2) | |
| Reverse | AAATGATGTGTGTGCTGGGT |
| Exon 20: | |
| (SEQ ID NO: 3) | |
| Forward | AGCAGCTCACTGACCAGATGT; |
| (SEQ ID NO: 4) | |
| Reverse | ACTCACTGCCATGCAGTGGA. |

PCR products were subjected to direct sequencing at Genewiz (Cambridge Mass., USA).

### Data Analysis

Determination of the Chalkley score was done as described (Fox et al. J. Pathol., 177: 275-283, 1995; Fox et al. Breast Cancer Res. Treat., 36: 219-226, 1995). Briefly, the three most vascular areas (hot spots) with the highest number of microvessel profiles in each tumor were photographed under an Olympus light microscope at 200×; a digital mask representing the Chalkley grid area, 0.196 mm², was used to count the CD31-positive spots in a blind fashion and the mean value of the three grid counts obtained. A two-sided t-test was used to determine significance.

### Targeted Mass Spectrometry (LC/MS/MS) for Compound A PK Study

Metabolites from 100 mg of mouse tumor samples were extracted using 80% methanol according to Yuan et al. (Nat. Protoc., 7: 872-881, 2012) 10 μL were injected and analyzed using a 5500 QTRAP hybrid triple quadrupole mass spectrometer (AB/SCIEX) coupled to a Prominence UFLC HPLC system (Shimadzu) via selected reaction monitoring (SRM) for the Q1/Q3 transition of 410.8/367.0 for 5-(2,6-dimorpholinopyrimidin-4-yl)-4-(trifluoromethyl)pyridin-2-amine (Compound A). ESI voltage was +4900V in positive ion mode using a dwell time of 4 msec and collision energy of 45. Approximately 15 data points were obtained for Compound A per LC/MS/MS experiment. Samples were delivered to the MS via hydrophilic interaction chromatography (HILIC) using a 4.6 mm i.d×10 cm Amide Xbridge column (Waters) at 350 μL/min. Gradients were run starting from 85% buffer B (HPLC grade acetonitrile) to 42% B from 0-5 minutes; 42% B to 0% B from 5-16 minutes; 0% B was held from 16-24 minutes; 0% B to 85% B from 24-25 minutes; 85% B was held for 7 minutes to re-equilibrate the column. Compound A eluted at approximately 3.50 min. Buffer A was comprised of 20 mM ammonium hydroxide/20 mM ammonium acetate (pH=9.0) in 95:5 water:acetonitrile. Peak areas from the total ion current for the Compound A metabolite SRM transition was integrated using MultiQuant v2.0 software (AB/SCIEX). For the concentration curve data, Compound A was prepared at concentrations of 1 nM, 10 nM, 100 nM, 500 nM, 1 μM and 10 μM in 40% methanol. 5 μL of each sample were injected using the parameters described above.

### Example 2

### Activation of the PI3K Pathway in BRCA1-Related Breast Cancer

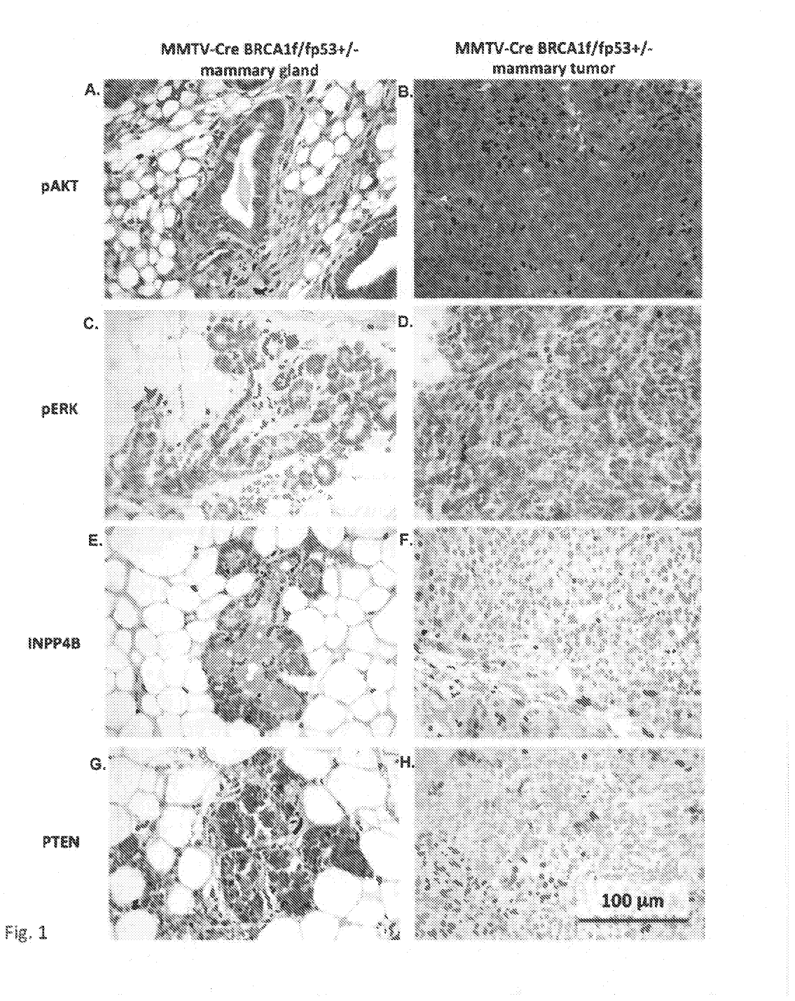

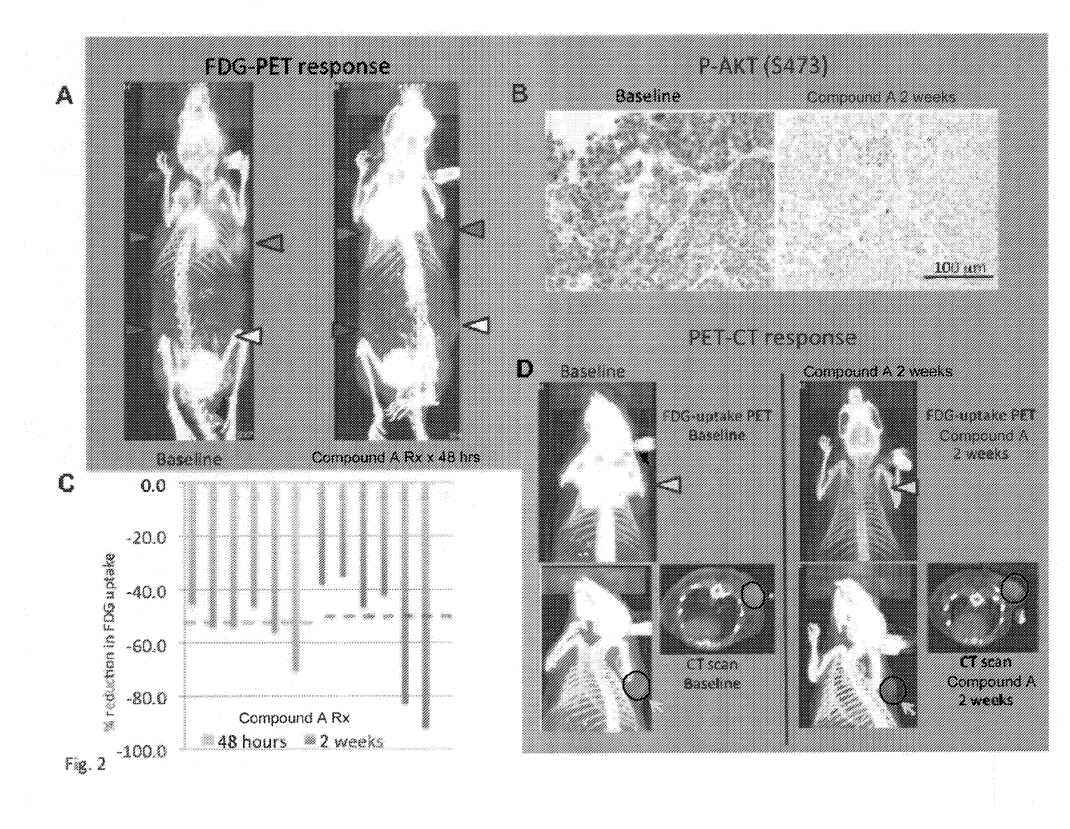

We and others have previously shown that the MMTV-CreBRCA1^{f/f}p53+/− mouse model faithfully recapitulates many aspects of human BRCA1-related breast cancer, including emergence on a background of multiple synchronous hyperproliferative lesions, high proliferative activity, absence of estrogen receptor expression and presence of EGFR-overexpression (Burga et al. Breast Cancer Res., 13: R30, 2011; Xu et al. Nat. Genet., 22: 37-43, 1999; Brodie et al. Oncogene, 20: 7514-7523, 2001; Shukla et al. Cancer Res., 66: 7151-7157, 2006). BRCA1 has been shown to suppress AKT (Xiang et al. Cancer Res., 68: 10040-10044, 2008) and ERK-activation in response to estrogen or EGF stimulation (Razandi et al. Mol. Cell. Biol., 24: 5900-5913, 2004; Yan et al. J. Biol. Chem., 277: 33422-33430, 2002) in cell based studies, suggesting that tumors with defects in BRCA1 might have an increase in AKT and/or ERK-phosphorylation. Consistent with these studies and with our prior data with this mouse model we found that phosphorylation of AKT at Serine 473 was strongly positive in both the cytoplasm and the nucleus in these tumor cells (FIG. 1B), while in the normal adjacent tissue cytoplasmic AKT phosphorylation was only seen in the basal layer of cells, not in luminal cells (FIG. 1A). Similarly, ERK phosphorylation was absent in normal mammary epithelial cells (FIG. 1C), while cytoplasmic ERK phosphorylation was seen in a majority, but not in all tumor cells (FIG. 1D).

Loss of function of PTEN, either through epigenetic silencing or through gross genomic loss, correlates with loss of function of BRCA1 in TNBC (Saal et al. Nat. Genet., 40: 102-107, 2008). Recently, Gewinner et al. (Cancer Cell., 16:115-125, 2009) as well as Fedele et al. (Proc. Natl. Acad. Sci. USA., 107:22231-22236, 2010) showed that, similar to PTEN, the tumor suppressor phosphatase INPP4B is lost in approximately 60% of TNBC, including BRCA1-related breast cancers. Consistent with these data in human disease, INPP4B and PTEN expression were strong in normal glands of MMTV-CreBRCA1^{f/f}p53+/− females (FIGS. 1E and 1G), but lost in tumor tissues (FIGS. 1F and 1H).

To examine whether activating PIK3CA mutations are responsible for the strong and uniform activation of AKT, we sequenced the PIK3CA gene of 11 murine BRCA1-deleted breast tumors. Consistent with the rarity of mutations in human TNBC, we found no activating hotspot mutations in exons 9 or 20 of PI3K. In human TNBC, activating mutations in PIK3CA are relatively rare and seen in only 8% of TNBC, confirming that the activation of the PI3K pathway in TNBC is mostly driven by regulatory mechanisms such as loss of PTEN and INPP4B, rather than by activating mutations in PIK3CA.

Collectively, these observations suggest that the MMTV-CreBRCA1^{f/f}p53+/− mouse model accurately recapitulates the activation of growth factor signaling seen in human BRCA1-related breast cancer, including activation of the PI3K and MAPK pathways and the absence of activating PI3K mutations. Based on this data, we decided to study whether inhibition of PI3K would be an effective treatment for BRCA1-related breast cancer.

### Example 3

### Pharmacodynamics of PI3K Inhibition in BRCA1-Related Breast Cancer

TNBCs, including the BRCA1 related subtype, exhibit high rates of glucose uptake, as judged by positron emission tomography (PET) using the radioactive glucose analog, ¹⁸F-fluorodeoxyglucose (FDG) (Specht et al. Clinical Cancer Research, 16: 2803-2810, 2010; Tafreshi et al. Cancer Control, 17: 143-55, 2010). Consistent with these observations in humans, we found that BRCA1-deleted tumors in our mouse model were highly avid for FDG. Tumors of sub-centimeter size were easily visualized using this technique (FIG. 2A). In a previous study (Engelman et al. Nat. Med., 14: 1351-1556, 2008) mouse lung tumors that resulted from transgenic expression of the H1047R mutant of PIK3CA were found to have high rates of glucose uptake as judged by FDG-PET, and the PI3K/mTOR inhibitor BEZ235 caused a reduction in the FDG-PET signal within two days, consistent with the known role of PI3K in regulating glucose uptake and glycolysis (Engelman et al. Nat. Rev. Genet., 7: 606-319, 2006; Vander Heiden. Sci. Transl. Med., 2: 31, 2010; Schnell et al. Cancer Res., 68: 6598-6607, 2008). We found that within 48 hours of instituting treatment with 5-(2,6-dimorpholinopyrimidin-4-yl)-4-(trifluoromethyl)pyridin-2-amine (Compound A), tumors in all treated animals showed a median decrease in FDG-uptake by 46.7% (range 38.1-92.3), which was sustained after 2 weeks of continued treatment with Compound A (median decrease by 54%, range 45.5-70.5%) and corresponded to inhibition of akt phosphorylation (FIGS. 2A-2D). These results indicate that activation of the PI3K pathway contributes to the up-regulation of glucose metabolism in BRCA1-related breast cancers and that oral delivery of Compound A results in inhibition of this response. Further evidence that Compound A inhibits PI3K signaling in the BRCA1 defective tumors was provided by the observation that phosphorylation of the downstream protein kinase, AKT at Ser-473 was strongly decreased in tumors treated with Compound A (FIG. 2B). It was remarkable that all BRCA1-related tumors examined showed a decrease in FDG-uptake and a decrease in AKT-phosphorylation in response to Compound A (FIGS. 2A, 2C, and 2D), suggesting that a high level of PI3K signaling and the consequent enhanced glucose metabolism is a common event in tumors that result from loss of BRCA-1 function. In addition, our data suggest that inhibition of FDG-uptake may be an early and predictive pharmacodynamic marker for response to treatments with PI3K inhibitors.

### Example 4

### Compound A PI3K Inhibitor Exerts Anti-Angiogenic Activity

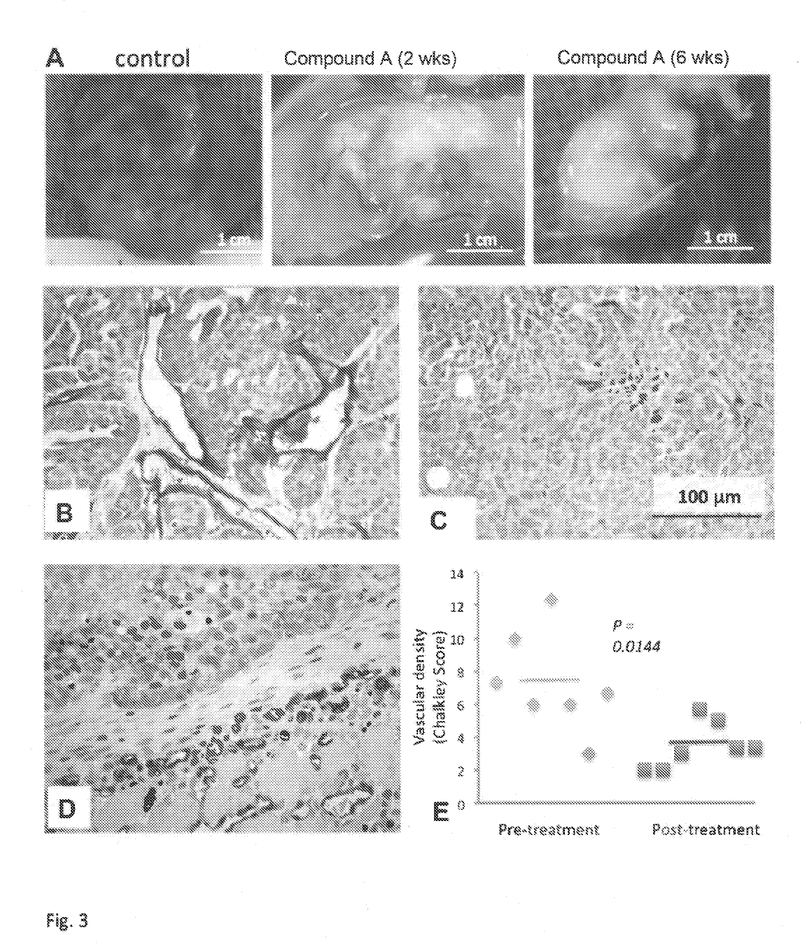

Tumor growth requires neo-vascularization of the expanding neoplastic tissue. The organized proliferation of vascular endothelial cells required for this process depends on growth factor signaling through receptor tyrosine kinases such as VEGFR1-3, TIE-1/2, FGFR1-2, PDGFR-β, and ERBB1-4, whose intracellular signaling is transduced by PI3K. It was previously shown that BEZ235, a PI3K-inhibitor with activity against PI3Kα and mTOR, inhibits the sprouting of new blood vessels in tumors, and disrupts the integrity of existing blood vessels (Schnell et al. Cancer Res., 68: 6598-6607, 2008; Yuan et al. Proc. Natl. Acad. Sci. USA., 105: 9739-9744, 2008). Spontaneous tumors in MMTV-CreBRCA1^{f/f}p53+/− mice grow rapidly, and are highly vascular (FIGS. 3A, left panel, and 3B). However after treatment with 5-(2,6-dimorpholinopyrimidin-4-yl)-4-(trifluoromethyl)pyridin-2-amine (Compound A), the gross pathology of tumors was notable for central pallor and, eventually, central necrosis (FIG. 3A, middle panel). In contrast, blood vessels in the tumor capsule remained initially intact, or became ectatic. Consistently, the tumor microvasculature, as visualized with an anti-CD31 stain, was diminished in response to Compound A (FIG. 3C) while it was maintained in the tumor capsule (FIG. 3D). The necrotic center of treated tumors was frequently hemorrhagic, indicating disorganized collapse of the tumor vasculature. We used the Chalkley count of CD31-positive microvessels (Fox et al. J. Pathol., 177: 275-283, 1995) to compare the vascularization before and after treatment with Compound A and found that both the size and number of blood vessels were starkly reduced in treated tumors (FIG. 3E). Thus, consistent with prior observations with BEZ235 (Yuan et al. Proc. Natl. Acad. Sci. USA., 105: 9739-9744, 2008), our data confirm that Compound A's anti-tumor activity is, in part, due to its anti-angiogenic activity, and thus this drug may have preferential activity in rapidly growing, endocrine-resistant tumors with a high degree of tumor angiogenesis.

### Example 5

### Effects of PI3K Inhibition on Compensatory Pathways in Tumor Cells

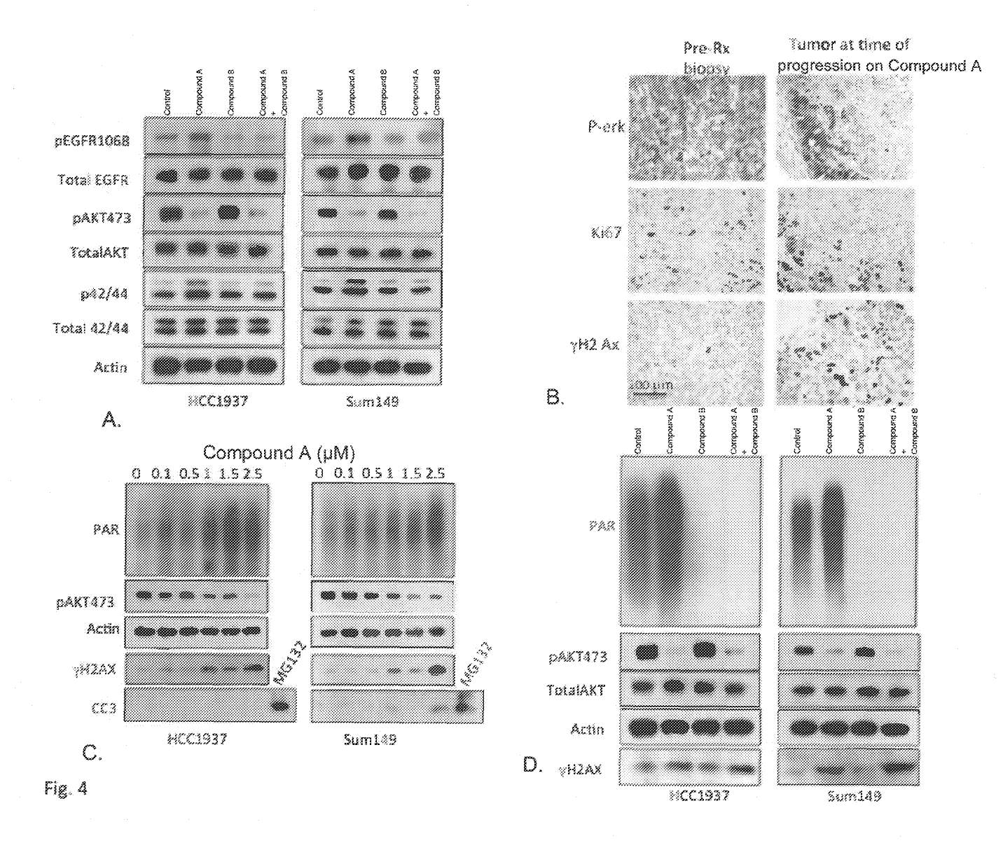

The upregulation of compensatory pathways in response to tumor cell treatments with inhibitors of mitogenic signaling is now a well-known phenomenon (Chandarlapaty et al. Cancer Cell, 19: 58-71, 2011). Consistent with these prior observations, we found that Compound A induced a compensatory activation of the EGFR/MAPK pathways in the human BRCA1 mutant breast cancer cell lines, HCC1937 (BRCA1 5382C mutation and homozygous deletion of PTEN and p53) (Tomlinson et al. Cancer Res., 58: 3237-3242, 1998), and SUM149 (BRCA1 2288delT, PTEN WT, p53 mutant) (Forozan et al. Br. J. Cancer, 81: 1328-1334, 1999; Saal et al. Nat. Genet., 40: 102-107, 2008) (FIG. 4A, second lane for each cell line). As expected, treatments with the PARP inhibitor Olaparib (Compound B) alone did not have a discernible effect on the activation status of EGFR, AKT, or MAPK (FIG. 4A, third lane for each cell line). However, with the combination treatment (FIG. 4A, last lane), we found that compensatory activation of EGFR and MAPK could be blocked by the addition of Compound B. These data suggest that PARP inhibition in tumor cells either restricts mitogenic signaling to PI3K-mediated signaling, or disables mechanisms that would re-route mitogenic signaling via EGFR/ERK when PI3K is inhibited.

### Example 6

### Treatments with Compound A Increase Indicators of DNA Damage but Decrease Rad51 Recruitment to Repair Foci

Loss of BRCA1 function results in genome instability due to defects in DNA repair by homologous recombination. As a consequence, BRCA1^{-/-} cells have high rates of DNA damage and are sensitized to the inhibition of alternative DNA repair mechanisms involving PARP-dependent poly(ADP)-ribosylation (Farmer et al. Nature, 434: 917-21, 2005). We examined the possibility that the high sensitivity of BRCA1 mutant tumors to PI3K pathway inhibitors is a consequence of a role for the PI3K pathway in maintaining cell survival during DNA repair or in facilitating DNA repair mechanisms. These experiments were carried out in vivo (FIG. 4B) and with the human BRCA1-mutant cell lines, HCC1937 and SUM149. We first examined the effect of Compound A on DNA repair responses in cells grown on plastic. Surprisingly, we found that in both cell lines H2AX phosphorylation on Serine 139 (γH2AX, a marker for DNA double-stranded damage) increased with increasing concentrations of Compound A and that this correlated with diminishing phosphorylation of AKT (FIG. 4C). Similarly, tumors treated with Compound A in vivo showed a substantial increase in the percentage of cells that express γH2AX (FIG. 4B).

Tumors with loss of BRCA1 rely on PARP-dependent poly-ADP-ribosylation (PAR) of key proteins involved in DNA damage repair (Farmer et al. Nature, 434: 917-21, 2005). Given the surprising increase in H2AX phosphorylation, we examined if treatment with Compound A would also affect PARP activity. Treatment with Compound A caused a dose dependent increase in overall poly-ADP-ribosylation (PAR) that paralleled the increase in H2AX phosphorylation and the decrease in AKT phosphorylation (FIG. 4C). Importantly, this increase in PAR was initially not accompanied by apoptotic cell death, as cells remained negative for cleaved caspase 3 (CC3) (FIG. 4C). The basal and Compound A-enhanced PAR could be completely blocked by treatment with the PARP inhibitor, Compound B (FIG. 4D), while γH2AX accumulation was enhanced with the combination of Compound A and Compound B (FIG. 4D).

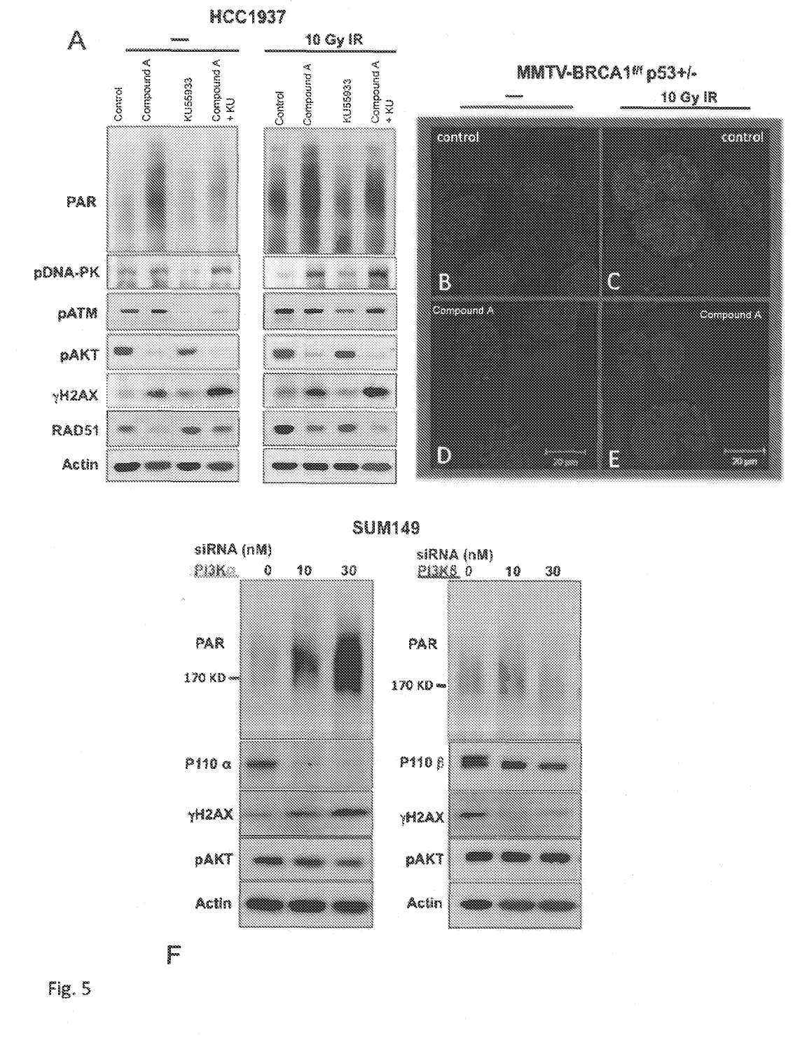

Thus, we observed that PI3K inhibition caused a significant increase in activities indicative of both types of DNA damage: PARP activity, which is required for base excision (BER) and single strand break (SSB) repair, as well as H2AX phosphorylation, indicative of the presence of DNA double strand breaks (DSBs). As H2AX is a substrate for the PI3K-related kinases (PIKKs) ATM and DNA-PK, we asked if Compound A had an effect on these kinases that would explain our findings. We examined PAR and γH2AX accumulation in HCC1937 cells in the absence and presence of the ATM-inhibitor KU-55933 (Hickson et al. Cancer Res., 64: 9152-9159, 2004) and monitored the response to ionizing radiation. As expected, KU-55933 led to a decrease in auto-phosphorylation of ATM (FIG. 5A, third and fourth lane of each panel) and prevented the increase in H2AX phosphorylation seen in response to ionizing radiation. However, KU-55933 did not prevent the Compound A-induced induction of γH2AX, which was robust both at baseline and in response to ionizing radiation (FIG. 5A, last lane of each panel), suggesting that an alternative kinase, such as DNA-PK is phosphorylating H2AX in response to PI3K inhibition. As shown in FIG. 5A, we found a strong increase in auto-phosphorylation of DNA-PK in response to addition of Compound A that corresponds to H2AX phosphorylation. These results clearly show that Compound A is not acting through an off-target inhibition of ATM or DNA-PK and suggest that inhibition of PI3K by Compound A leads to activation of DNA-PK through a yet unknown mechanism.

Consistent with the results in FIG. 4C, we found that the PAR accumulation in the presence Compound A alone increased (FIG. 5A, left panel, second lane). In the presence of the combination of Compound A and KU-55933 PAR accumulation was attenuated but still greater than in the control, suggesting that the Compound A-induced increase in PAR was only partially offset by inhibition of ATM, again consistent with an ATM-independent mechanism for PAR accumulation and its induction by PI3K inhibition.

To determine if PI3K inhibition affected the assembly of DNA damage repair foci, we examined the ability of tumor cells from our mouse model to recruit Rad51 to DNA damage repair foci (FIG. 5B-5E), following a protocol established previously (Drost et al. Cancer Cell, 20: 797-809, 2011). We generated cell cultures from tumors of MMTV-CreBRCA1^{f/f}p53+/− mice and examined their ability to form DNA repair foci 6 hours after exposure to ionizing radiation (10 Gy). We found that there was residual double-strand repair activity as shown by the formation of Rad51 foci in this mouse model with a hypomorphic exon 11 deletion (FIG. 5C). Surprisingly, the formation of Rad51 foci in response to ionizing radiation was completely blocked by pre-treatment of these cells with Compound A (FIG. 5D). A similar phenomenon was observed in HCC1937 cells: while ionizing radiation induced accumulation of Rad51 and H2AX phosphorylation as reported previously (Yuan et al. Cancer Res., 59: 3547-3551, 1999) (FIG. 5A, control lanes), pre-treatment with the PI3K inhibitor Compound A led to a dissociation of this radiation response as we saw a failure to increase Rad51, but a prominent augmentation of radiation-induced H2AX phosphorylation in the presence of Compound A (FIG. 5A, second lane of each panel). The mechanism by which Compound A decreases Rad51 recruitment to repair foci is yet unknown. However, this observation of a defective DSB repair response may, at least in part, provide an additional explanation for the in vivo synergy of PARP inhibition and PI3K inhibition.

### Example 7

### Effects of Compound A are Specific for PI3Kα Inhibition

Given the unanticipated and striking effects of the Compound A-class PI3K inhibitor, Compound A, on the DNA damage response, we asked if these effects were specific to a single Class I PI3K isoform, required inhibition of multiple PI3Ks, or could be an off-target effect of Compound A. In the BRCA1-mutant cell line SUM149 down-regulation of PI3Kα, but not PI3Kβ, with siRNA led to a stark increase in phosphorylation of DNA-PK, H2AX (γH2AX), and poly-(ADP)ribosylation (PAR), as well as a stark decrease in Rad51 accumulation (FIG. 5F). These data confirm that it is the inhibition of PI3Kα that is decisive for the disruption of the DNA damage response in these cells.

### Example 8

### Synergistic Effect of PI3K Inhibitor and PARP Inhibitor in the Treatment of Breast Cancer

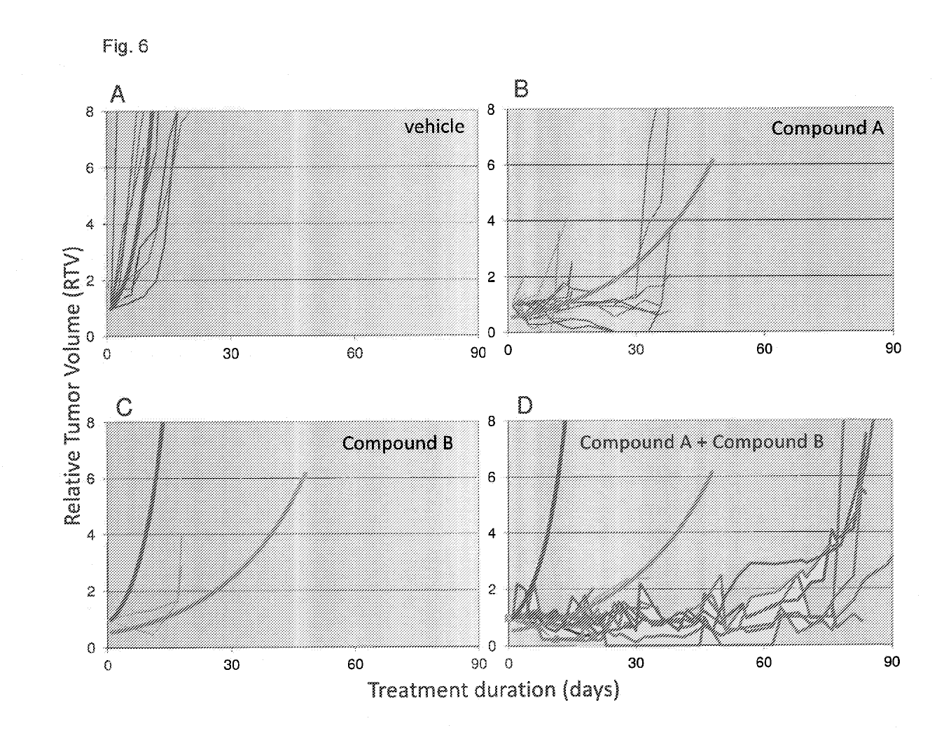

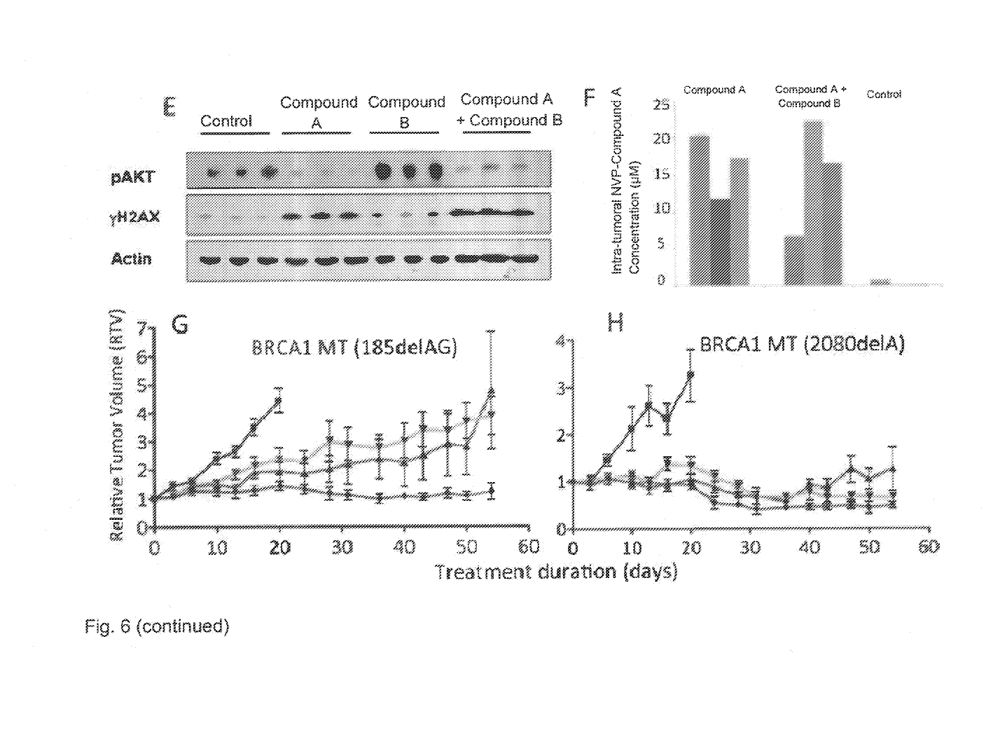

We next compared the effects of Compound A and Compound B as single agents and the combination of both drugs on tumor growth. Female virgin MMTV-CreBRCA1^{f/f}p53+/− mice were observed for the development of spontaneous tumors, which typically occurs at age 8-12 months. Once tumors reached a diameter of 5-7 mm, mice were randomized to either vehicle control treatments, treatments with Compound A via oral gavage, Compound B intraperitoneally, or the combination of Compound A with Compound B, all once a day continuously. An initial set of mice was treated with Compound A at 50 mg/kg/day, alone or in combination with Compound B (50 mg/kg/day) and a second set at Compound A 30 mg/kg/day alone or in combination with Compound B (50 mg/kg/day). No significant difference was seen with regard to efficacy or p-AKT-suppression between the two dose levels of Compound A and data were pooled (FIGS. 6A-6D). Tumors were measured at least 3 times a week, and relative tumor volume, as a ratio to baseline tumor volume, was calculated for each treatment modality (FIGS. 6A-6D). Trendlines (bold lines) were determined based on the best fit to the data in vehicle control and Compound A only. Once tumors were established, their doubling time was rapid if treated with vehicle only, on average 5 days (FIG. 6A). Treatments with Compound A alone significantly prolonged tumor doubling time by a factor of 5 (26 days versus 5 days, FIG. 6B), however, tumors eventually grew (FIG. 6B). In this mouse model, tumor growth was delayed three-fold with the use of Compound B (tumor doubling time 16 days versus 5 days, FIG. 6C). When Compound B and Compound A were combined, we found a surprising in vivo synergistic activity, with a tumor doubling time of over 70 days, a 140-fold increase over control (leftmost bold trendline).

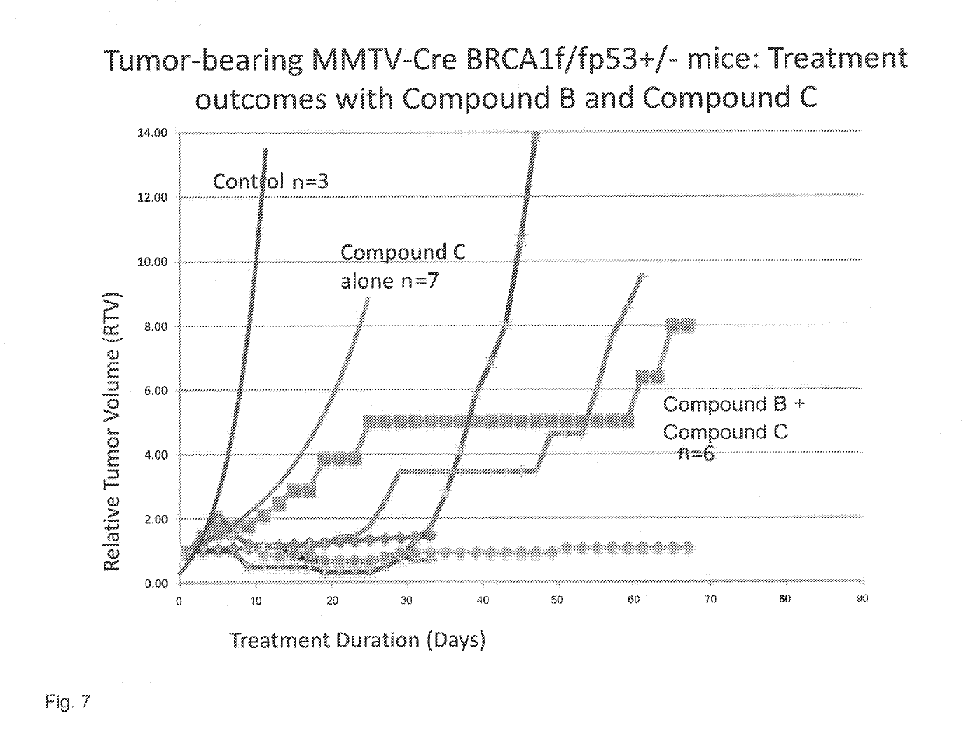

In order to determine whether the contribution of PI3K inhibition was a result of the inhibition of, specifically, PI3Kα, as suggested by our data in FIG. 5F, we performed analogous experiments with tumor-bearing MMTV-CreBRCA1^{f/f}p53+/− mice in which the mice were treated with a vehicle control, a PI3Kα-specific inhibitor (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), a pan-PARP inhibitor (Compound B), or both Compound B and Compound C, and tumor growth was subsequently measured as a function of median tumor doubling time (FIG. 7). We found that the combination of Compound B and Compound C exhibited surprising in vivo synergistic activity (FIG. 7, compare control or Compound C alone to each of the six independent Compound B+Compound C experiments), similar to that observed with Compound A and Compound B. These data confirm the importance of PI3Kα inhibition, in particular, in the synergistic effect observed when PARP function is also inhibited.

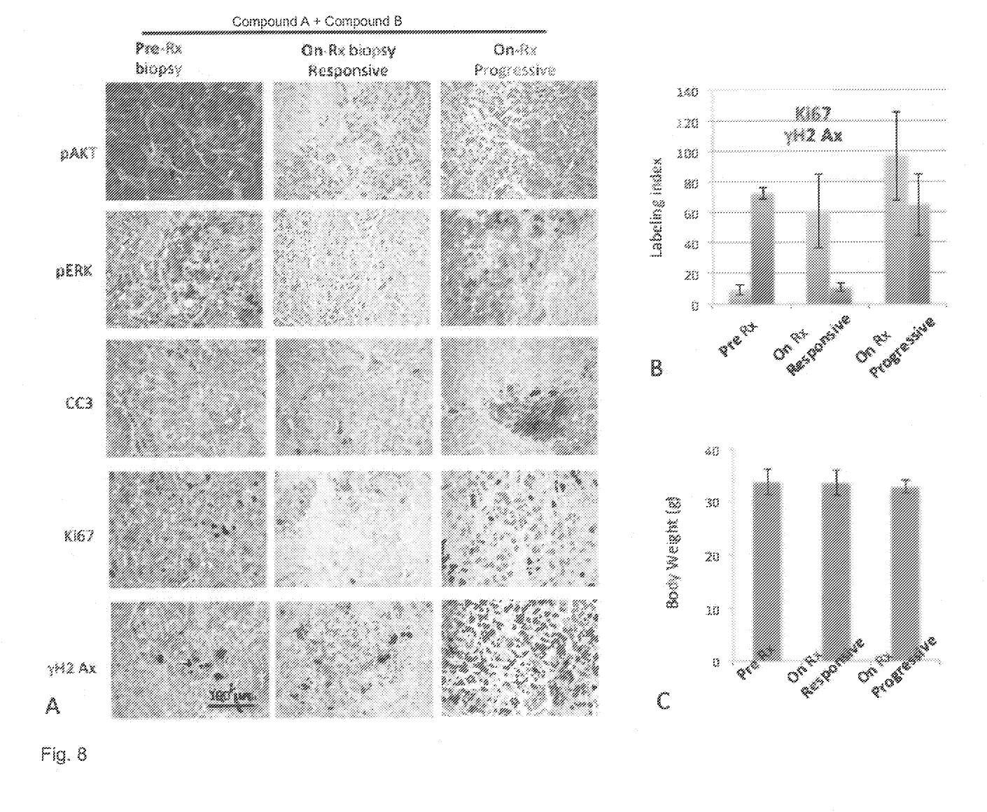

To ensure target inhibition, we also investigated the effect of PI3K inhibition on AKT phosphorylation. To this end, we obtained pre-treatment biopsies and matched tumor specimen within 2 hours of the last dose of Compound A and found that Compound A potently reduced AKT phosphorylation (FIG. 6E). The dual combination of Compound A and Compound B did not result in measurable toxicity, such as weight loss (FIG. 8C), even in mice that were treated for over 3 months. In tumor tissue lysates from the combination treatment, we observed inhibition of p-AKT with the combination treatment and induction of γH2AX (FIG. 6E), consistent with results observed in the in vitro studies with cell lines (FIG. 4). Interestingly, Compound B alone led to an induction of AKT phosphorylation in vivo (FIG. 6E), an observation consistent with an increased FDG-uptake in Compound B-treated tumors as opposed to Compound A or the combination, both of which strongly reduced FDG-uptake (FIG. 2).

In order to examine if there was a pharmacokinetic interaction between the PI3K and PARP inhibitors, we examined Compound A levels in animals treated with Compound A at 30 mg/kg/day and the combination of Compound A and Compound B (30 mg/kg/day and 50 mg/kg/day, respectively). For these studies, tissue extracts were processed for Mass Spectrometry 3 hours after the last dose. We found that while Compound A levels in tumor tissues were variable, they were consistently in the micro-molar range and were not affected by concurrent administration of Compound B (FIG. 6F). The mouse model used here for BRCA1-related breast cancer, MMTV-CreBRCA1^{f/f}p53+/−, results in the residual expression of a hypomorphic BRCA1 protein, and we did find residual Rad51 recruitment to repair foci (FIG. 5C). To test the applicability of our results to human BRCA1-related breast cancer, we treated xenograft tumors established from patients with BRCA1-related breast cancer (FIGS. 6G and 6H). The first patient-derived tumor was derived from a patient with an N-terminal germline mutation in BRCA1 (185delAG). At the time of tissue acquisition, this tumor had developed resistance to standard chemotherapy as well as Compound B, which had been administered in the context of a clinical trial. Growth of this tumor in NOD/SCID mice was modestly attenuated by either Compound A or Compound B. However, the combination induced stability over a period of 8 weeks (FIG. 6G), confirming the in vivo synergy that we observed in our genetically engineered mouse model of BRCA1-related breast cancer (FIG. 6D). The second human tumor was derived from a patient with a C-terminal BRCA1 germline mutation (2080delA). The patient who donated this tumor specimen had not yet been treated, and the tumor showed exquisite sensitivity to the Compound A, Compound B, and the combination of both drugs. These human ex vivo data confirm the sensitivity of BRCA1-related breast cancer to Compound A, Compound B, and their combination.

### Example 9

### Resistance to Treatments that Include PI3K Inhibitors Occurs at the “Pushing Margin” and is Associated with ERK Phosphorylation

Eventually, even in tumors that received dual treatments, resistance was observed, and at that point, tumors re-grew rapidly (FIGS. 6A-6D). To determine the nature of resistance to the Compound A and Compound B combination, we examined pre-treatment biopsies, on-treatment biopsies at the time of response on day 10 and post-treatment tissue at the time of progression (FIG. 8A). Target inhibition, i.e., suppression of AKT phosphorylation, was maintained even in resistant tumors (FIG. 8A, panel 1), suggesting that resistance to Compound A is not due to PI3K pathway activation but to relief of feedback inhibition of alternative pathways, including MAPK-activation as suggested earlier (Chandarlapaty et al. Cancer Cell, 19: 58-71, 2011). The “pushing margin”, i.e., a highly proliferative rim of tumor cells that rarely infiltrate the surrounding tissue is a hallmark of BRCA1-related tumors (Lakhani et al. J. Natl. Cancer Inst., 90: 1138-1145, 1998), yet its biological basis is not understood. Interestingly, we found an increase in the number of cells with high phospho-ERK levels especially at the “pushing margin” of the tumor, paralleled by an increase in proliferating, i.e., Ki67-positive cells (FIGS. 8A and 8B). This phenomenon, the concentration of p-ERK positive cells at the “pushing margin” was seen in tumors prior to treatment (FIGS. 4B and 8A), at the time of progression on Compound A alone (FIG. 4B) or at the time of progression on the combination of the Compound A with Compound B, while in responding tumors (day 10 biopsy) p-ERK positive cells were conspicuously absent (FIG. 8A). Of note, mice tolerated the combination treatment without significant weight loss (FIG. 8C). As expected with PI3K inhibition and consistent with the p-ERK status of tumor cells, we found that tumors initially showed a stark decrease in proliferative activity (Ki67, FIGS. 8A and 8B), and that resistant tumors were characterized by high mitotic activity (FIG. 4B; FIGS. 8A and 8B). Thus, activation of pro-proliferative MAPK-signaling may be a major driver for the resistance of tumors treated with PI3K-inhibitors.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All patents, patent applications, patent application publications, and other publications cited or referred to in this specification are herein incorporated by reference to the same extent as if each independent patent, patent application, patent application publication, or publication was specifically and individually indicated to be incorporated by reference. Such patent applications specifically include U.S. Provisional Patent Application Nos. 61/541,964 and 61/683,941, filed on Sep. 30, 2011 and Aug. 16, 2012, respectively, from which this application claims benefit.

1-60. (canceled)

61. A composition comprising a therapeutically effective amount of at least one phosphatidyl inositol 3 kinase (PI3K) inhibitor and at least one poly(ADP-ribose) polymerase (PARP) inhibitor.

62. The composition of claim 61, wherein the PI3K inhibitor is a small molecule.

63. The composition of claim 61, wherein the PI3K inhibitor is a PI3Kα-specific inhibitor.

64. The composition of claim 63, wherein the PI3Kα-specific inhibitor is selected from the group consisting of (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), 4-[2-(1H-indazol-4-yl)-6-[(4-methylsulfonylpiperazin-1-yl)methyl]thieno[3,2-d]pyrimidin-4-yl]morpholine (GDC-0941), (2S)-1-N-[5-(2-tert-butyl-1,3-thiazol-4-yl)-4-methyl-1,3-thiazol-2-yl]pyrrolidine-1,2-dicarboxamide (A66), N-[(E)-(6-bromoimidazo[1,2-a]pyridin-3-yl)methylideneamino]-N,2-dimethyl-5-nitrobenzenesulfonamide (PIK-75), N-(7,8-dimethoxy-2,3-dihydroimidazo[1,2-c]quinazolin-5-yl)pyridine-3-carboxamide (PIK-90), PWT33597, INK1117, and CNX-1351, or a pharmaceutically acceptable salt thereof.

65. The composition of claim 61, wherein the PI3K inhibitor is a pan-class I PI3K inhibitor.

66. The composition of claim 65, wherein the pan-class I PI3K inhibitor is a Compound A-class PI3K inhibitor.

67. The composition of claim 62, wherein the small molecule is selected from the group consisting of 5-(2,6-dimorpholinopyrimidin-4-yl)-4-(trifluoromethyl)pyridin-2-amine (Compound A), (S)-pyrrolidine-1,2-dicarboxylic acid 2-amide 1-[(2-tert-butyl-4′-methyl-[4,5]bithiazolyl-2′-yl)-amide] (A66 S), [(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5-h]isochromen-10-yl]acetate (PX-866), 4-[2-(1H-indazol-4-yl)-6-[(4-methylsulfonylpiperazin-1-yl)methyl]thieno[3,2-d]pyrimidin-4-yl]morpholine (GDC-0941), N-[3-(2,1,3-benzothiadiazol-5-ylamino)quinoxalin-2-yl]-4-methylbenzenesulfonamide (XL147), and 2-[(6-aminopurin-9-yl)methyl]-5-methyl-3-(2-methylphenyl)quinazolin-4-one (IC87114), or a pharmaceutically acceptable salt thereof.

68. The composition of claim 61, wherein the PARP inhibitor is a small molecule.

69. The composition of claim 68, wherein the small molecule PARP inhibitor is selected from the group consisting of 4-[[3-[4-(cyclopropanecarbonyl)piperazine-1-carbonyl]-4-fluorophenyl]methyl]-2H-phthalazin-1-one (Compound B, i.e., Olaparib), 4-iodo-3-nitrobenzamide (Iniparib), 2-[(2R)-2-methylpyrrolidin-2-yl]-1H-benzimidazole-4-carboxamide (ABT-888), 8-Fluoro-2-{4-[(methylamino)methyl]phenyl}-1,3,4,5-tetrahydro-6H-azepino[5,4,3-cd]indol-6-one (AG014699), 4-methoxy-carbazole (CEP 9722), 2-[4-[(3S)-piperidin-3-yl]phenyl]indazole-7-carboxamide hydrochloride (MK 4827), and 3-aminobenzamide, or a pharmaceutically acceptable salt thereof.

70. The composition of claim 61, wherein the at least one PI3K inhibitor is (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), or a pharmaceutically acceptable salt thereof, and the at least one PARP inhibitor is 4-[[3-[4-(cyclopropanecarbonyl)piperazine-1-carbonyl]-4-fluorophenyl]methyl]-2H-phthalazin-1-one (Compound B), or a pharmaceutically acceptable salt thereof.

71. The composition of claim 61, wherein the at least one PI3K inhibitor is 5-(2,6-dimorpholinopyrimidin-4-yl)-4-(trifluoromethyl)pyridin-2-amine (Compound A), or a pharmaceutically acceptable salt thereof, and the at least one PARP inhibitor is 4-[[3-[4-(cyclopropanecarbonyl)piperazine-1-carbonyl]-4-fluorophenyl]methyl]-2H-phthalazin-1-one (Compound B), or a pharmaceutically acceptable salt thereof.

72. A method of treating a subject having a proliferative disease comprising administering to the subject a therapeutically effective amount of the composition of claim 61.

73. The method of claim 72, wherein a predisposition to responsiveness of the subject to treatment with the composition is determined by detecting an alteration in a germline BRCA1 gene, PTEN expression, BSA1 expression, Akt phosphorylation, and/or H2AX phosphorylation from a sample from the subject relative to a sample from a control subject.

74. A method of treating a subject having a proliferative disease comprising the step of administering to the subject a therapeutically effective amount of at least one PI3K inhibitor and at least one PARP inhibitor in an amount sufficient to treat the subject.

75. The method of claim 74, wherein the PI3K inhibitor and the PARP inhibitor are administered together in the same composition.

76. The method of claim 74, wherein the PI3K inhibitor and the PARP inhibitor are administered separately.

77. The method of claim 74, wherein the PI3K inhibitor is a small molecule.

78. The method of claim 74, wherein the PI3K inhibitor is a PI3Kα-specific inhibitor.

79. The method of claim 78, wherein the PI3Kα-specific inhibitor is selected from the group consisting of (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), 4-[2-(1H-indazol-4-yl)-6-[(4-methylsulfonylpiperazin-1-yl)methyl]thieno[3,2-d]pyrimidin-4-yl]morpholine (GDC-0941), (2S)-1-N-[5-(2-tert-butyl-1,3-thiazol-4-yl)-4-methyl-1,3-thiazol-2-yl]pyrrolidine-1,2-dicarboxamide (A66), N-[(E)-(6-bromoimidazo[1,2-a]pyridin-3-yl)methylideneamino]-N,2-dimethyl-5-nitrobenzenesulfonamide (PIK-75), N-(7,8-dimethoxy-2,3-dihydroimidazo[1,2-c]quinazolin-5-yl)pyridine-3-carboxamide (PIK-90), PWT33597, INK1117, and CNX-1351, or a pharmaceutically acceptable salt thereof.

80. The method of claim 74, wherein the PI3K inhibitor is a pan-class I PI3K inhibitor.

81. The method of claim 80, wherein the PI3K inhibitor is a Compound A-class PI3K inhibitor.

82. The method of claim 77, wherein the small molecule is selected from the group consisting of 5-(2,6-dimorpholinopyrimidin-4-yl)-4-(trifluoromethyl)pyridin-2-amine (Compound A), (S)-pyrrolidine-1,2-dicarboxylic acid 2-amide 1-[(2-tert-butyl-4′-methyl-[4,5′]bithiazolyl-2′-yl)-amide] (A66 S), [(3aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5-Nisochromen-10-yl]acetate (PX-866), 4-[2-(1H-indazol-4-yl)-6-[(4-methylsulfonylpiperazin-1-yl)methyl]thieno[3,2-d]pyrimidin-4-yl]morpholine (GDC-0941), N-[3-(2,1,3-benzothiadiazol-5-ylamino)quinoxalin-2-yl]-4-methylbenzenesulfonamide (XL147), and 2-[(6-aminopurin-9-yl)methyl]-5-methyl-3-(2-methylphenyl)quinazolin-4-one (IC87114), or a pharmaceutically acceptable salt thereof.

83. The method of claim 74, wherein the at least one PARP inhibitor is a small molecule.

84. The method of claim 83, wherein the small molecule PARP inhibitor is selected from the group consisting of 4-[[3-[4-(cyclopropanecarbonyl)piperazine-1-carbonyl]-4-fluorophenyl]methyl]-2H-phthalazin-1-one (Compound B, i.e., Olaparib), 4-iodo-3-nitrobenzamide (Iniparib), 2-[(2R)-2-methylpyrrolidin-2-yl]-1H-benzimidazole-4-carboxamide (ABT-888), 8-Fluoro-2-{4-[(methylamino)methyl]phenyl}-1,3,4,5-tetrahydro-6H-azepino[5,4,3-cd]indol-6-one (AG014699), 4-methoxy-carbazole (CEP 9722), 2-[4-[(3S)-piperidin-3-yl]phenyl]indazole-7-carboxamide hydrochloride (MK 4827), and 3-aminobenzamide, or a pharmaceutically acceptable salt thereof.

85. The method of claim 74, wherein the at least one PI3K inhibitor is (2S)—N1-(4-methyl-5-(2-(1,1,1-trifluoro-2-methylpropan-2-yl)pyridin-4-yl)thiazol-2-yl)pyrrolidine-1,2-dicarboxamide (Compound C), or a pharmaceutically acceptable salt thereof, and the at least one PARP inhibitor is 4-[[3-[4-(cyclopropanecarbonyl)piperazine-1-carbonyl]-4-fluorophenyl]methyl]-2H-phthalazin-1-one (Compound B), or a pharmaceutically acceptable salt thereof.

86. The method of claim 74, wherein the at least one PI3K inhibitor is 5-(2,6-dimorpholinopyrimidin-4-yl)-4-(trifluoromethyl)pyridin-2-amine (Compound A), or a pharmaceutically acceptable salt thereof, and the at least one PARP inhibitor is 4-[[3-[4-(cyclopropanecarbonyl)piperazine-1-carbonyl]-4-fluorophenyl]methyl]-2H-phthalazin-1-one (Compound B), or a pharmaceutically acceptable salt thereof.

87. The method of claim 74, wherein a predisposition to responsiveness of the subject to treatment is determined by detecting an alteration in a germline BRCA1 gene, PTEN expression, BSA1 expression, Akt phosphorylation, and/or H2AX phosphorylation from a sample from the subject relative to a sample from a control subject.


SEQUENCE LISTING (4 sequences)

SEQ ID NO: 1 - DNA, 21 bp, Artificial Sequence
  Exon 9 Forward Primer
cgcatacctgcatctgttcta

SEQ ID NO: 2 - DNA, 20 bp, Artificial Sequence
  Exon 9 Reverse Primer
aaatgatgtgtgtgctgggt

SEQ ID NO: 3 - DNA, 21 bp, Artificial Sequence
  Exon 20 Forward Primer
agcagctcactgaccagatgt

SEQ ID NO: 4 - DNA, 20 bp, Artificial Sequence
  Exon 20 Reverse Primer
actcactgccatgcagtgga